(19) United States
(12) Patent Application Publication
(10) Pub. No.: US 2025/0281419 A1
(43) Pub. Date: Sep. 11, 2025

(54) LIPID AMINES

(71) Applicant: ModernaTX, Inc., Cambridge, MA (US)

(72) Inventors: Mohindra Seepersaud, Acton, MA (US); Matthew Crawford, Cambridge, MA (US); Daniel Tate, Somerville, MA (US); Mark Cornebise, Arlington, MA (US); Christopher Karl McLaughlin, Medford, MA (US); Ana Cadete Pires, Arlington, MA (US)

(21) Appl. No.: 18/705,140

(22) PCT Filed: Oct. 28, 2022
(86) PCT No.: PCT/US2022/048231

§ 371 Date: Apr. 26, 2024

Related U.S. Application Data
(60) Provisional application No. 63/273,441, filed on Oct. 29, 2021.

(51) Int. Cl. A61K 9/51; C07J 9/00; C07J 43/00

(52) U.S. Cl. CPC A61K 9/5123; A61K 9/5192; C07J 9/005; C07J 43/003

(57) ABSTRACT

The present invention relates to lipid amine compounds of formula (A1) which are useful in the preparation of lipid nanoparticle compositions for delivery of therapeutic or prophylactic payload into cells.

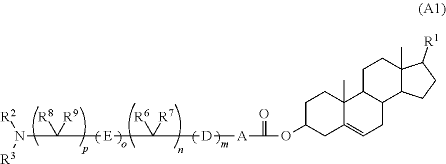

### CROSS-REFERENCE TO RELATED APPLICATIONS

This application is a U.S. National application of PCT/US2022/048231, filed Oct. 28, 2022, which claims the benefit of the filing date of U.S. Provisional Application No. 63/273,441, filed Oct. 29, 2021, the contents of each of which are incorporated by reference in its entirety herein.

### FIELD OF THE INVENTION

The present invention relates to lipid amine compounds which are useful in the preparation of lipid nanoparticle compositions for delivery of therapeutic or prophylactic payload into cells.

### BACKGROUND OF THE INVENTION

The delivery of biologically active payloads, such as nucleic acids and proteins, to cells has the potential to be used to treat a variety of diseases and/or conditions. However, effective targeted delivery of such payloads represents a continuing medical challenge. In particular, the delivery of nucleic acids to cells is made difficult by the relative instability and low cell permeability of such species.

Lipid nanoparticles provided an effective transport vehicle for the payloads into cells and intracellular compartments, but improvements in safety, efficacy, and specificity are still needed. Thus, there exists a need to develop lipid nanoparticle compositions to facilitate the delivery of therapeutics and prophylactics, such as nucleic acids, into cells.

### SUMMARY OF THE INVENTION

Provided herein is a lipid amine having the structure of Formula A1:

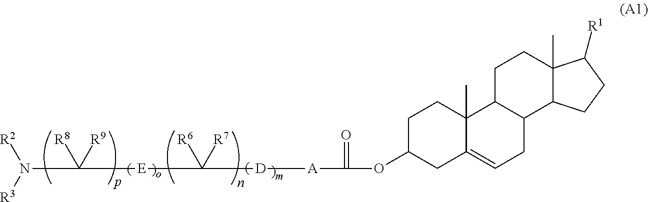

or a salt thereof, wherein constituent members are defined herein.

Also provided herein is a lipid nanoparticle composition comprising a lipid amine of Formula A1, or a salt thereof.

Also provided herein is a pharmaceutical composition comprising a pharmaceutically acceptable carrier and a lipid nanoparticle composition comprising a lipid amine of Formula A1, or a salt thereof.

Also provided herein is a method of delivering a payload into a cell comprising contacting the cell with a lipid nanoparticle composition of the invention.

Also provided herein is a method of delivering a therapeutic or prophylactic payload to a patient comprising administering a lipid nanoparticle composition of the invention to the patient.

Also provided herein is a process of preparing a lipid nanoparticle composition, the process comprising combining the lipid amine compound of any one of claims 1-39, or a salt thereof, with one or more additional lipids selected from:
(i) an ionizable lipid,
(ii) a phospholipid,
(iii) a structural lipid, and
(iv) optionally a PEG-lipid.

Also provided herein is a product of any of the processes described herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### DETAILED DESCRIPTION

Provided herein is a lipid amine having the structure of Formula A1:

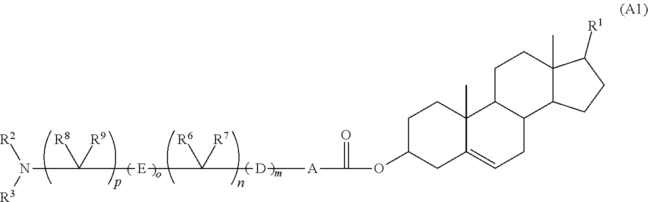

or a salt thereof, wherein:
A is NR^{a} or CR⁴R⁵;
D is O or S—S;
E is C(O), C(O)NH, or O;
R¹ is C₁₋₁₄ alkyl, C₁₋₁₄ alkenyl, or C₁₋₁₄ hydroxyalkyl;
R² and R³ are each independently selected from H, methyl, and ethyl, wherein the methyl or ethyl is optionally substituted by OH;
or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR₈R₉, OH, and halo;
or R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, OH, and halo;
R^{a} is H or methyl;
R⁴, R⁵, R⁶, R⁷, R⁸, R⁹, and R¹⁰ are each independently selected from H and C₁₋₄ alkyl;
or R⁴ and R⁵ together with the carbon atom to which they are attached form a C₃₋₅ cycloalkyl group;
or R⁶ and R⁷ together with the carbon atom to which they are attached form a C₃₋₅ cycloalkyl group;
or R⁸ and R⁹ together with the carbon atom to which they are attached form a C₃₋₅ cycloalkyl group;
m is 0 or 1;
n is 0, 1, 2, 3, 4, or 5;
is 0 or 1; and
p is 0, 1, 2, 3, 4, 5, 6, 7, 8, 9, 10, 11, or 12;
wherein at least one of m, n, o, and p is other than 0;
wherein p is 1 when R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, OH, and halo; and
wherein when m is 1, then A is CR⁴R⁵ and n is 1.

In some embodiments, the compound is other than:

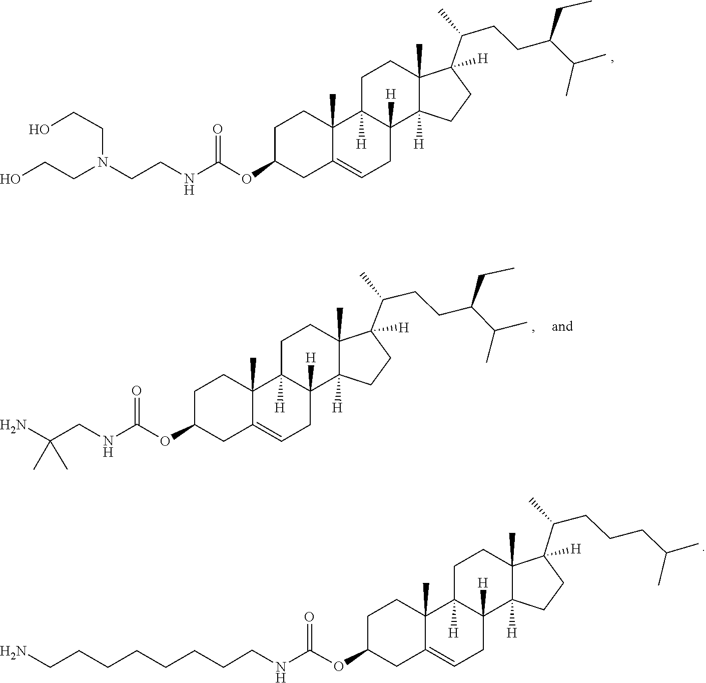

In some embodiments, the compound is other than:

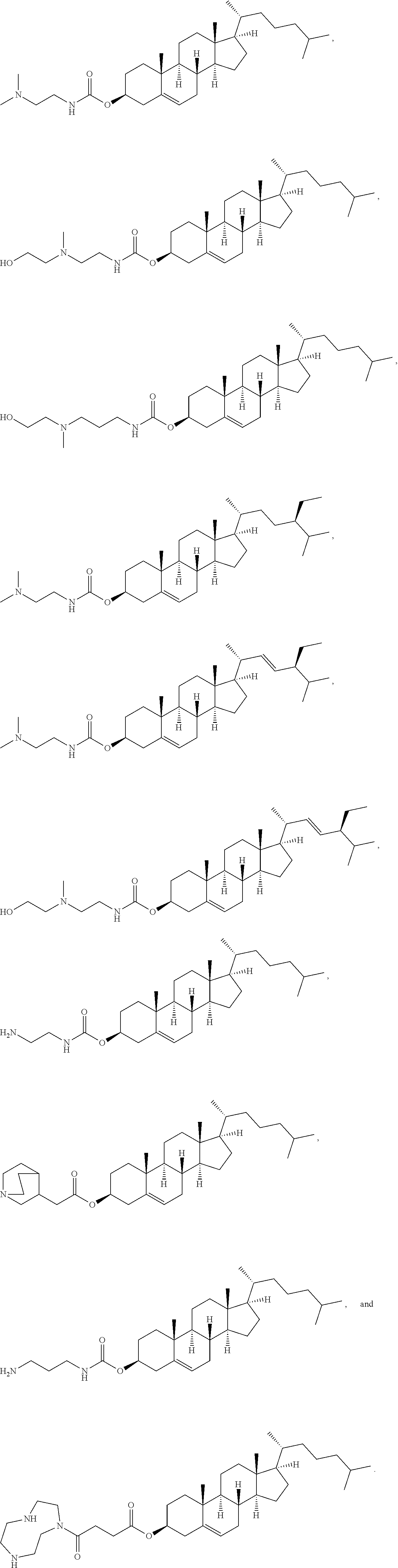

In some embodiments, R¹ is C₁₋₁₄ alkyl. In some embodiments, R¹ is C₃₋₁₂ alkyl. In some embodiments, R¹ is C₆₋₁₂ alkyl. In some embodiments, R¹ is C₈₋₁₀ alkyl. In some embodiments, R¹ is C₈ alkyl. In some embodiments, R¹ is C₁₀ alkyl.

In some embodiments, R¹ is C₁₋₁₄ hydroxyalkyl. In some embodiments, R¹ is C₃₋₁₂ hydroxyalkyl. In some embodiments, R¹ is C₆₋₁₂ hydroxyalkyl. In some embodiments, R¹ is C₈₋₁₀ hydroxyalkyl. In some embodiments, R¹ is C₈ hydroxyalkyl. In some embodiments, R¹ is C₁₀ hydroxyalkyl.

In some embodiments, R¹ is C₁₋₁₄ alkenyl. In some embodiments, R¹ is C₃₋₁₂ alkenyl. In some embodiments, R¹ is C₆₋₁₂ alkenyl. In some embodiments, R¹ is C₈₋₁₀ alkenyl. In some embodiments, R¹ is C₈ alkenyl. In some embodiments, R¹ is C₁₀ alkenyl.

In some embodiments, R¹ is

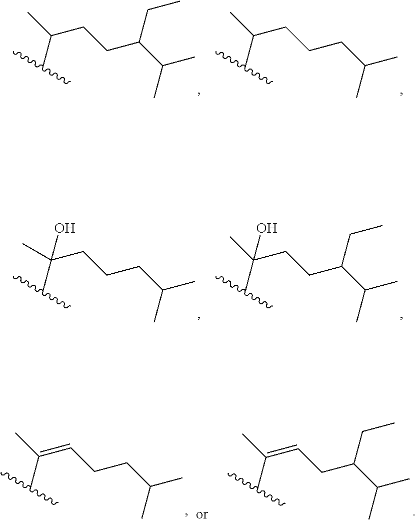

In some embodiments, R¹ is

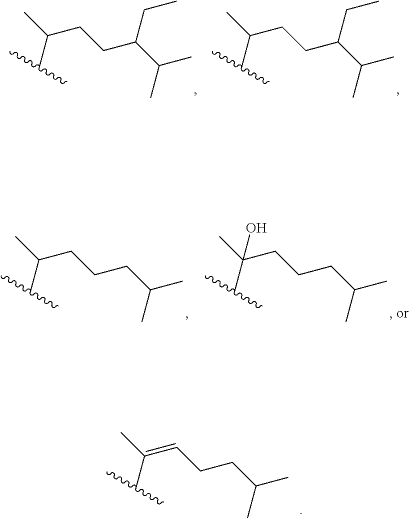

In some embodiments, R¹ is

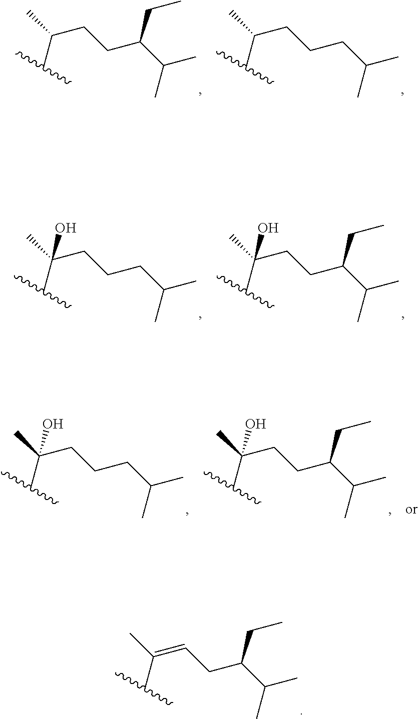

In some embodiments, R¹ is

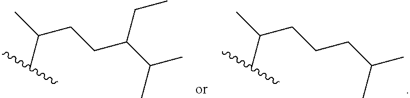

In some embodiments, R1 is

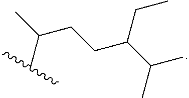 [SMILES: *C(C)CCC(CC)C(C)C]

In some embodiments, R1 is

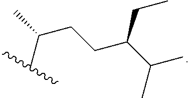 [SMILES: *[C@H](C)CC[C@@H](CC)C(C)C]

In some embodiments, A is NR^{a}. In some embodiments, A is CR⁴R⁵.

In some embodiments, R^{a} is H. In some embodiments, R^{a} is methyl.

In some embodiments, R⁴ and R⁵ are both H. In some embodiments, R⁴ and R⁵ are both C₁₋₄ alkyl. In some embodiments, R⁴ and R⁵ are both methyl. In some embodiments, one of R⁴ and R⁵ is H and the other of R⁴ and R⁵ is C₁₋₄ alkyl. In some embodiments, one of R⁴ and R⁵ is H and the other of R⁴ and R⁵ is methyl. In some embodiments, R⁴ and R⁵ together with the carbon atom to which they are attached form a C₃₋₅ cycloalkyl group. In some embodiments, R⁴ and R⁵ together with the carbon atom to which they are attached form a C₃ cycloalkyl group. In some embodiments, at least one R⁴ is C₁₋₄ alkyl. In some embodiments, at least one R⁴ is methyl.

In some embodiments, m is 0. In some embodiments, m is 1.

In some embodiments, D is O. In some embodiments, D is S—S.

In some embodiments, n is 0. In some embodiments, n is 1. In some embodiments, n is 2. In some embodiments, n is 3. In some embodiments, n is 4. In some embodiments, n is 5. In some embodiments, n is 0, 1, or 2.

In some embodiments, R⁶ and R⁷ are both H. In some embodiments, R⁶ and R⁷ are both C₁₋₄ alkyl. In some embodiments, R⁶ and R⁷ are both methyl. In some embodiments, one of R⁶ and R⁷ is H and the other of R⁶ and R⁷ is C₁₋₄ alkyl. In some embodiments, one of R⁶ and R⁷ is H and the other of R⁴ and R⁵ is methyl. In some embodiments, R⁶ and R⁷ together with the carbon atom to which they are attached form a C₃₋₅ cycloalkyl group. In some embodiments, R⁶ and R⁷ together with the carbon atom to which they are attached form a C₃ cycloalkyl group. In some embodiments, at least one R⁶ is C₁₋₄ alkyl. In some embodiments, at least one R⁶ is methyl.

In some embodiments, o is 0. In some embodiments, o is 1.

In some embodiments, E is C(O)NH. In some embodiments, E is O. In some embodiments, E is C(O).

In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 1, 2, 3, 4, 6, 8, or 10. In some embodiments, p is 2, 6, 8, or 10.

In some embodiments, R⁸ and R⁹ are both H. In some embodiments, R⁸ and R⁹ are both C₁₋₄ alkyl. In some embodiments, R⁸ and R⁹ are both methyl. In some embodiments, one of R⁸ and R⁹ is H and the other of R⁸ and R⁹ is C₁₋₄ alkyl. In some embodiments, one of R⁸ and R⁹ is H and the other of R⁸ and R⁹ is methyl. In some embodiments, R⁸ and R⁹ together with the carbon atom to which they are attached form a C₃₋₅ cycloalkyl group. In some embodiments, R⁸ and R⁹ together with the carbon atom to which they are attached form a C₃ cycloalkyl group. In some embodiments, at least one R⁸ is C₁₋₄ alkyl. In some embodiments, at least one R⁸ is methyl.

In some embodiments, n is 1, R⁶ is H, and R⁷ is H. In some embodiments, n is 2 and both R⁶ and R⁷ are H. In some embodiments, p is 1, R⁸ is C₁₋₄ alkyl, and R⁹ is C₁₋₄ alkyl. In some embodiments, p is 1, R⁸ is methyl, and R⁹ is methyl. In some embodiments, p is 1 and R⁸ and R⁹ together with the carbon atom to which they are attached form a C₃₋₅ cycloalkyl group. In some embodiments, p is 1 and R⁸ and R⁹ together with the carbon atom to which they are attached form a C3 cycloalkyl group. In some embodiments, p is 2 and each R⁸ and R⁹ are H. In some embodiments, p is 3 and each R⁸ and R⁹ are H. In some embodiments, p is 4 and each R⁸ and R⁹ are H. In some embodiments, p is 6 and each of R⁸ and R⁹ are H. In some embodiments, p is 8 and each R⁸ and R⁹ are H. In some embodiments, p is 10 and each R⁸ and R⁹ are H.

In some embodiments, m is 0, n is 0, o is 0, and p is 2. In some embodiments, m is 0, n is 0, o is 0, and p is 3. In some embodiments, m is 0, n is 0, o is 0, and p is 4. In some embodiments, m is 0, n is 0, o is 0, and p is 8. In some embodiments, m is 0, n is 0, o is 0, and p is 10. In some embodiments, m is 0, n is 1, o is 0, and p is 1. In some embodiments, m is 0, n is 2, o is 1, and p is 2. In some embodiments, m is 1, n is 1, o is 1, and p is 2. In some embodiments, m is 1, n is 1, o is 1, and p is 6. In some embodiments, m is 1, n is 1, o is 1, and p is 8. In some embodiments, m is 1, n is 1, o is 1, and p is 10.

In some embodiments, m is 0, n is 0, o is 0, p is 2, and each R⁸ and R⁹ are H. In some embodiments, m is 0, n is 0, o is 0, p is 3, and each R⁸ and R⁹ are H. In some embodiments, m is 0, n is 0, o is 0, p is 4, and each R⁸ and R⁹ are H. In some embodiments, m is 0, n is 0, o is 0, p is 8, and each R⁸ and R⁹ are H. In some embodiments, m is 0, n is 0, o is 0, p is 10, and each R⁸ and R⁹ are H. In some embodiments, m is 0, n is 1, R⁶ is H, R⁷ is H, o is 0, p is 1, R⁸ is C₁₋₄ alkyl, and R⁹ is C₁₋₄ alkyl. In some embodiments, m is 0, n is 1, R⁶ is H, R is H, o is 0, p is 1, R⁸ is methyl, and R⁹ is methyl. In some embodiments, m is 0, n is 1, R⁶ is H, R⁷ is H, o is 0, p is 1, R⁸ and R⁹ together with the carbon atom to which they are attached form a C₃₋₅ cycloalkyl group. In some embodiments, m is 0, n is 1, R⁶ is H, R⁷ is H, o is 0, p is 1, R⁸ and R⁹ together with the carbon atom to which they are attached form a C3 cycloalkyl group. In some embodiments, m is 0, n is 2, each of R⁶ and R⁷ are H, o is 1, E is O, p is 2, and each of R⁸ and R⁹ are H. In some embodiments, m is 1, n is 1, R⁶ is H, R⁷ is H, o is 1, E is C(O)NH, p is 2, and each of R⁸ and R⁹ are H. In some embodiments, m is 1, n is 1, R⁶ is H, R⁷ is H, o is 1, E is C(O)NH, p is 6, and each of R⁸ and R⁹ are H. In some embodiments, m is 1, n is 1, R⁶ is H, R⁷ is H, o is 1, E is C(O)NH, p is 8, and each of R⁸ and R⁹ are H. In some embodiments, m is 1, n is 1, R⁶ is H, R⁷ is H, o is 1, E is C(O)NH, p is 10, and each of R⁸ and R⁹ are H.

In some embodiments, m is 0, n is 0, o is 0, p is 1, and R⁸ with R² and R³ together with the atoms to which they are attached and any intervening atoms, form a 7-18 membered bridged heterocycloalkyl group and R⁹ is H. In some embodiments, m is 0, n is 0, o is 0, p is 1, and R⁸ with R² and R³ together with the atoms to which they are attached and any intervening atoms, form a 7-12 membered bridged heterocycloalkyl group and R⁹ is H. In some embodiments, m is 0, n is 0, o is 0, p is 1, and R⁸ with R² and R³ together with the atoms to which they are attached and any intervening atoms, form a 8 membered bridged heterocycloalkyl group and R⁹ is H. In some embodiments, m is 0, n is 0, o is 0, p is 1, and R⁹ is H and R⁸ with R² and R³ together with the atoms to which they are attached and any intervening atoms, form a 7-18 membered bridged heterocycloalkyl group having the formula:

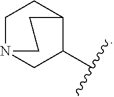 [SMILES: *C1CN2CCC1CC2]

In some embodiments, R² and R³ are both H. In some embodiments, R² and R³ are both methyl. In some embodiments, R² and R³ are both methyl substituted by OH. In some embodiments, R² and R³ are both ethyl. In some embodiments, R² and R³ are both ethyl substituted by OH.

In some embodiments, one of R² and R³ is H and the other of R² and R³ is methyl. In some embodiments, one of R² and R³ is H and the other of R² and R³ is methyl substituted with OH. In some embodiments, one of R² and R³ is H and the other of R² and R³ is ethyl. In some embodiments, one of R² and R³ is H and the other of R² and R³ is ethyl substituted with OH.

In some embodiments, one of R² and R³ is methyl and the other is ethyl. In some embodiments, one of R² and R³ is methyl substituted with OH and the other of R² and R³ is ethyl. In some embodiments, one of R² and R³ is methyl and the other of R² and R³ is ethyl substituted with OH. In some embodiments, one of R² and R³ is methyl substituted with OH and the other of R² and R³ is ethyl substituted with OH.

In some embodiments, both R² and R³ are

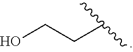 [SMILES: *CCO]

In some embodiments, one of R² and R³ is methyl and the other of R² and R³ is

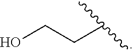 [SMILES: *CCO]

In some embodiments, R², R³, and R⁸, together with the atoms to which they are attached and any intervening atoms, form a 7-18 membered bridged heterocycloalkyl group. In some embodiments, R², R³, and R⁸, together with the atoms to which they are attached and any intervening atoms, form a 7-12 membered bridged heterocycloalkyl group. In some embodiments, R², R³, and R⁸, together with the atoms to which they are attached and any intervening atoms, form an 8 membered bridged heterocycloalkyl group. In some embodiments, R², R³, and R⁶, together with the atoms to which they are attached and any intervening atoms, form a 7-18 membered bridged heterocycloalkyl group having the formula:

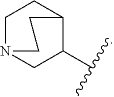 [SMILES: *C1CN2CCC1CC2]

In some embodiments, R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form a 7-12 membered heterocycloalkyl group comprising 1, 2, or 3 ring-forming NR¹⁰ groups, wherein the 7-12 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form a 8-10 membered heterocycloalkyl group comprising 1, 2, or 3 ring-forming NR¹⁰ groups, wherein the 8-10 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form a 8-10 membered heterocycloalkyl group comprising 1, 2, or 3 ring-forming NCH₃ or NH groups, wherein the 8-10 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form an 8-10 membered heterocycloalkyl group comprising 1, 2, or 3 ring-forming NCH₃ or NH groups.

In some embodiments, R² and R³ together with the N atom to which they are attached form a heterocycloalkyl group of formula:

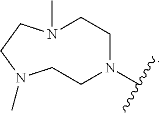 [SMILES: *N1CCN(C)CCN(C)CC1]

In some embodiments:
A is NR^{a} or CR⁴R⁵;
D is S—S;
E is C(O), C(O)NH, or O;
R¹ is C₁₋₁₄ alkyl;
R² and R³ are each independently selected from H, methyl, and ethyl substituted by OH;
or R² and R³ together with the N atom to which they are attached form a 7-18 membered heterocycloalkyl group comprising two ring-forming NR¹⁰ groups;
or R², R³, and R⁸, together with the atoms to which they are attached and any intervening atoms, form a 7-18 membered bridged heterocycloalkyl group;
R^{a} is H;
R⁴, R⁵, R⁶, and R⁷ are each H;
R⁸ and R⁹ are each independently selected from H and C₁₋₄ alkyl;
or R⁸ and R⁹ together with the carbon atom to which they are attached form a C₃₋₅ cycloalkyl group;
R¹⁰ is C₁₋₄ alkyl;
m is 0 or 1;
n is 0, 1, or 2;
o is 0 or 1; and
p is 0, 1, 2, 3, 4, 6, 8, or 10,
wherein at least one of m, n, o, and p is other than 0;
wherein p is 1 when R², R³, and R⁸, together with the atoms to which they are attached and any intervening atoms, form a 7-18 membered bridged heterocycloalkyl group; and
wherein when m is 1, then A is CR⁴R⁵ and n is 1.

In some embodiments:
A is NR^{a} or CR⁴R⁵,
D is S—S;
E is C(O), C(O)NH, or O;
R¹ is C₁₋₁₄ alkyl;
R² and R³ are each independently selected from H, methyl, and ethyl substituted by OH;
or R² and R³ together with the N atom to which they are attached form a 7-18 membered heterocycloalkyl group comprising two ring-forming NR¹⁰ groups;
or R², R³, and R⁸, together with the atoms to which they are attached and any intervening atoms, form a 7-18 membered bridged heterocycloalkyl group;
R^{a} is H or methyl;
R⁴, R⁵, R⁶, and R⁷ are each H;
R⁸ and R⁹ are each independently selected from H and C₁₋₄ alkyl;
or R⁸ and R⁹ together with the carbon atom to which they are attached form a C₃₋₅ cycloalkyl group;
R¹⁰ is C₁₋₄ alkyl;
m is 0 or 1;
n is 0, 1, or 2;
o is 0 or 1; and
p is 0, 1, 2, 3, 4, 6, 8, or 10,
wherein at least one of m, n, o, and p is other than 0;
wherein p is 1 when R², R³, and R⁸, together with the atoms to which they are attached and any intervening atoms, form a 7-18 membered bridged heterocycloalkyl group; and
wherein when m is 1, then A is CR⁴R⁵ and n is 1.

In some embodiments, the compound of Formula A1 is a compound of Formula A2:

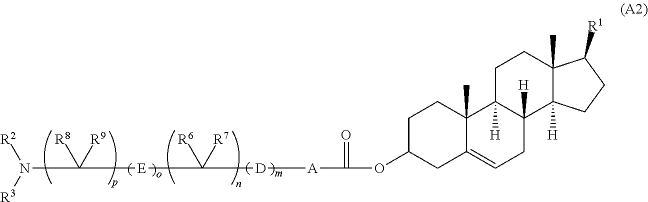

or salt thereof.

In some embodiments, the compound of Formula A1 is selected from:

| No. | Structure |
|---|---|
| SA44 | 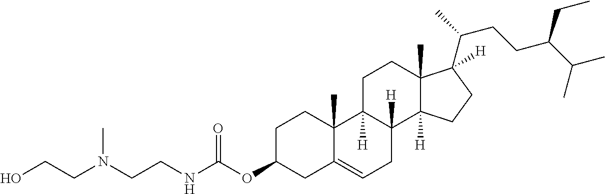 |
| SA45 | 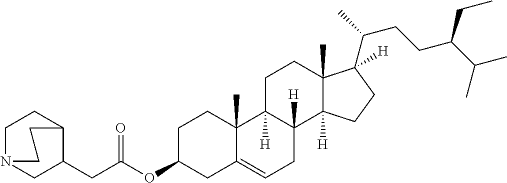 |
| SA46 | 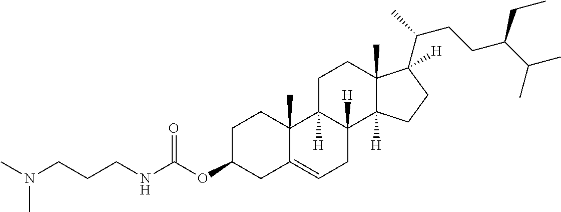 |
| SA47 | 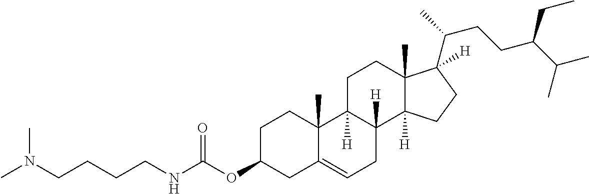 |
| SA49 | 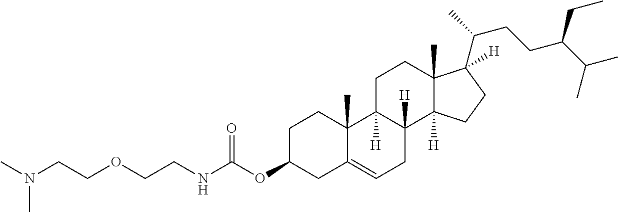 |
| SA52 | 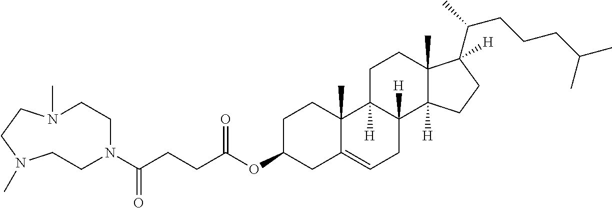 |
| SA53 | 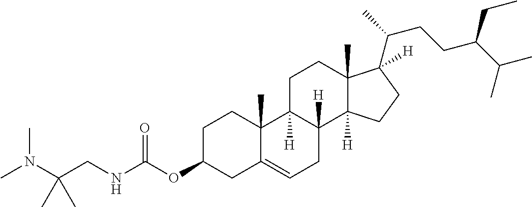 |
| SA54 | 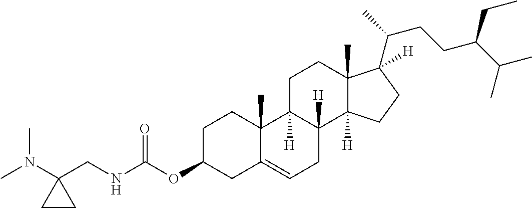 |
| SA86 | 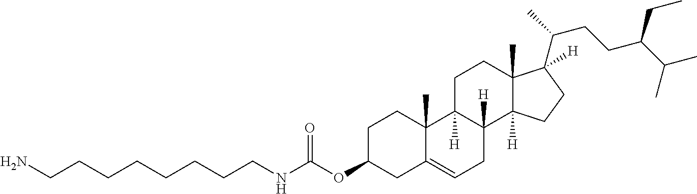 |
| SA91 | 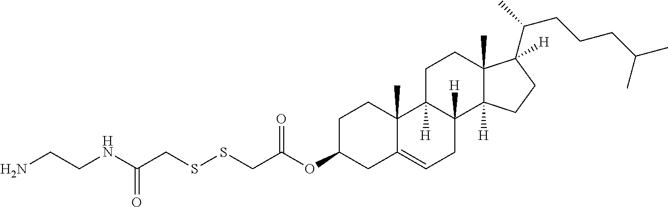 |
| SA92 | 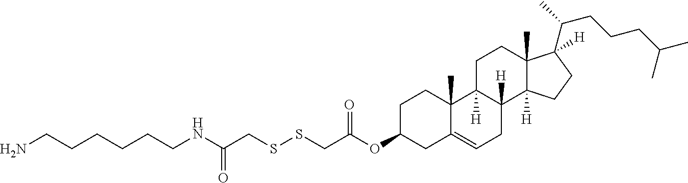 |
| SA93 | 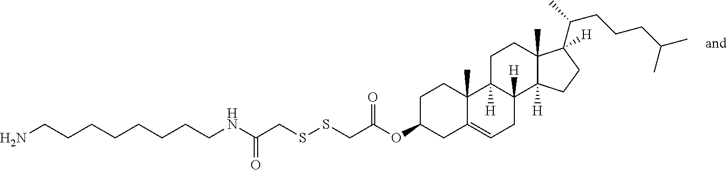 |
| SA94 | 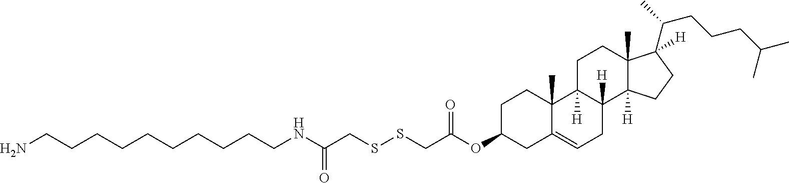 |

or salt of any of the aforementioned.

In some embodiments, the lipid amine of the present invention is a compound having the formula:

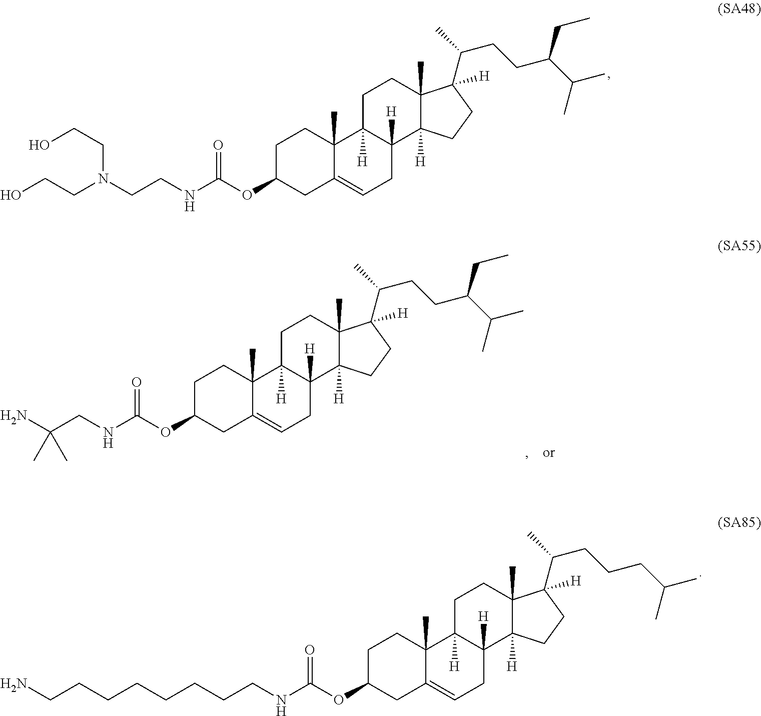

or salt thereof.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### Lipid Nanoparticle Compositions

The present invention further provides a lipid nanoparticle (LNP) composition comprising a lipid amine disclosed herein, such as a lipid amine of Formula A1. In some embodiments, the lipid nanoparticle composition further comprises, in addition to the lipid amine, at least one of an ionizable lipid, a phospholipid, a structural lipid, and a PEG-lipid. In some embodiments, the lipid nanoparticles of the lipid nanoparticle composition are loaded with payload. In some embodiments, the lipid amine is disposed primarily on the outer surface of the lipid nanoparticles of the lipid nanoparticle composition. In some embodiments, the lipid nanoparticle composition has a greater than neutral zeta potential at physiologic pH.

In some embodiments, the lipid nanoparticle composition of the present invention comprises:
(i) an ionizable lipid,
(ii) a phospholipid,
(iii) a structural lipid,
(iv) optionally a PEG-lipid,
(v) optionally a payload for delivery into a cell, and
(vi) a lipid amine as disclosed herein, such as the lipid amine of Formula A1.

The lipid nanoparticle compositions of the invention can further comprise additional components, including but not limited to, helper lipids, stabilizers, salts, buffers, and solvents. The helper lipid is a non-cationic lipid. The helper lipid may comprise at least one fatty acid chain of at least eight carbons and at least one polar headgroup moiety. In some embodiments, the lipid nanoparticle core has a neutral charge at a neutral pH.

In some embodiments, the weight ratio of the lipid amine to payload in the lipid nanoparticle compositions of the invention is about 0.1:1 to about 15:1, about 0.2:1 to about 10:1, about 1:1 to about 10:1, about 1:1 to about 8:1, about 1:1 to about 7:1, about 1:1 to about 6:1, about 1:1 to about 5:1, about 1:1 to about 4:1, or about 1.25:1 to about 3.75:1. In some embodiments, a weight ratio of the lipid amine to payload is about 1.25:1, about 2.5:1, or about 3.75:1. In some embodiments, a molar ratio of the lipid amine to payload is about 0.1:1 to about 20:1, about 1.5:1 to about 10:1, about 1.5:1 to about 9:1, about 1.5:1 to about 8:1, about 1.5:1 to about 7:1, about 1.5:1 to about 6:1, or about 1.5:1 to about 5:1. In some embodiments, a molar ratio of the lipid amine to payload is about 1.5:1, about 2:1, about 3:1, about 4:1, or about 5:1.

In some embodiments, the lipid nanoparticle composition of the invention is characterized as having a zeta potential of about 5 mV to about 20 mV. In some embodiments, the lipid nanoparticle composition has a zeta potential of about 5 mV to about 15 mV. In some embodiments, the lipid nanoparticle composition has a zeta potential of about 5 mV to about 10 mV. Zeta potential measures the surface charge of colloidal dispersions. The magnitude of the zeta potential indicates the degree of electrostatic repulsion between adjacent, similarly charged particles in the dispersion. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. This measurement is more sensitive and less stress inducing than ISO Method 13099-1:2012 which only uses one angle of detection and required higher voltage for operation. In some embodiments, the zeta potential of the herein described empty lipid nanoparticle compositions lipid is measured using an instrument employing the principle of MP-PALS. Zeta potential can be measured on a Malvern Zetasizer (Nano ZS).

In some embodiments, greater than about 80%, greater than about 90%, or greater than about 95% of the lipid amine is on the surface on the lipid nanoparticles of the lipid nanoparticle composition.

In some embodiments, the lipid nanoparticle composition has a polydispersity value of less than about 0.4, less than about 0.3 or less than about 0.2. In some embodiments, the LNP has a polydispersity value of about 0.1 to about 1, about 0.1 to about 0.5 or about 0.1 to about 0.3.

In some embodiments, the lipid nanoparticles of the lipid nanoparticle composition has a mean diameter of about 40 nm to about 150 nm, about 50 nm to about 100 nm, about 60 nm to about 120 nm, about 60 nm to about 100 nm, or about 60 nm to about 80 nm.

In some embodiments, a general polarization of laurdan of the lipid nanoparticles of the lipid nanoparticle composition is greater than or equal to about 0.6. In some embodiments, the LNP has a d-spacing of greater than about 6 nm or greater than about 7 nm.

In some embodiments, at least about 50%, at least about 75%, at least about 90%, at least about 95% of the lipid nanoparticles of the lipid nanoparticle composition have a surface fluidity value of greater than a threshold polarization level.

### Ionizable Lipid

As used herein, the term “ionizable lipid” has its ordinary meaning in the art and may refer to a lipid comprising one or more charged moieties. In some embodiments, an ionizable lipid may be positively charged or negatively charged. For instance, an ionizable lipid may be positively charged at lower pHs, in which case it could be referred to as “cationic lipid.” In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the LNP comprises about 30 mol % to about 60 mol %, about 35 mol % to about 55 mol %, about 40 mol % to about 50 mol %, or about 45 mol % to about 50 mol % of ionizable lipid.

In some embodiments, the ionizable lipid is an ionizable amino lipid. In one embodiment, the ionizable amino lipid may have a positively charged hydrophilic head and a hydrophobic tail that are connected via a linker structure.

In some embodiments, the ionizable lipid is a compound of Formula (I):

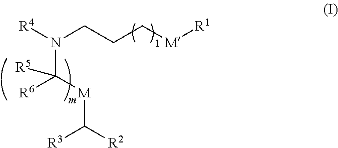

or an N-oxide or a salt thereof, wherein:
R¹ is

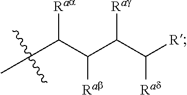

wherein

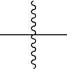

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and

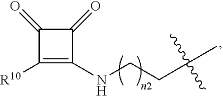

wherein n is selected from 1, 2, 3, 4, and 5;
wherein

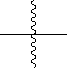

denotes a point of attachment,
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (I), or an N-oxide or a salt thereof, wherein:
R¹ is

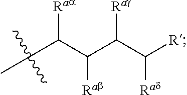

wherein

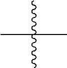

denotes a point of attachment;
R^{aα}, R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is a compound of Formula (I), or an N-oxide or a salt thereof, wherein:
R¹ is

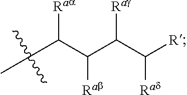

wherein

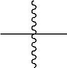

denotes a point of attachment;
R^{aα}, R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 3; and
m is 7.

In some embodiments, the ionizable lipid is a compound of Formula (I), or an N-oxide or a salt thereof, wherein:
R¹ is

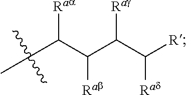

wherein

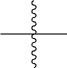

denotes a point of attachment;
R^{aα} is C₂₋₁₂ alkyl;
R^{aβ}, R^{aγ}, and R^{aδ} are each H;
R² and R³ are each C₁₋₁₄ alkyl;
R⁴ is

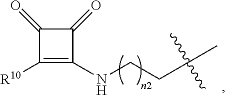

R¹⁰ is —NH(C₁₋₆ alkyl);
n2 is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is a compound of Formula (I), or an N-oxide or a salt thereof, wherein:
R¹ is

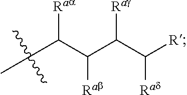

wherein

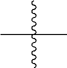

denotes a point of attachment;
R^{aα}, R^{aβ}, and R^{aδ} are each H;
R^{aγ} is C₂₋₁₂ alkyl;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is selected from:

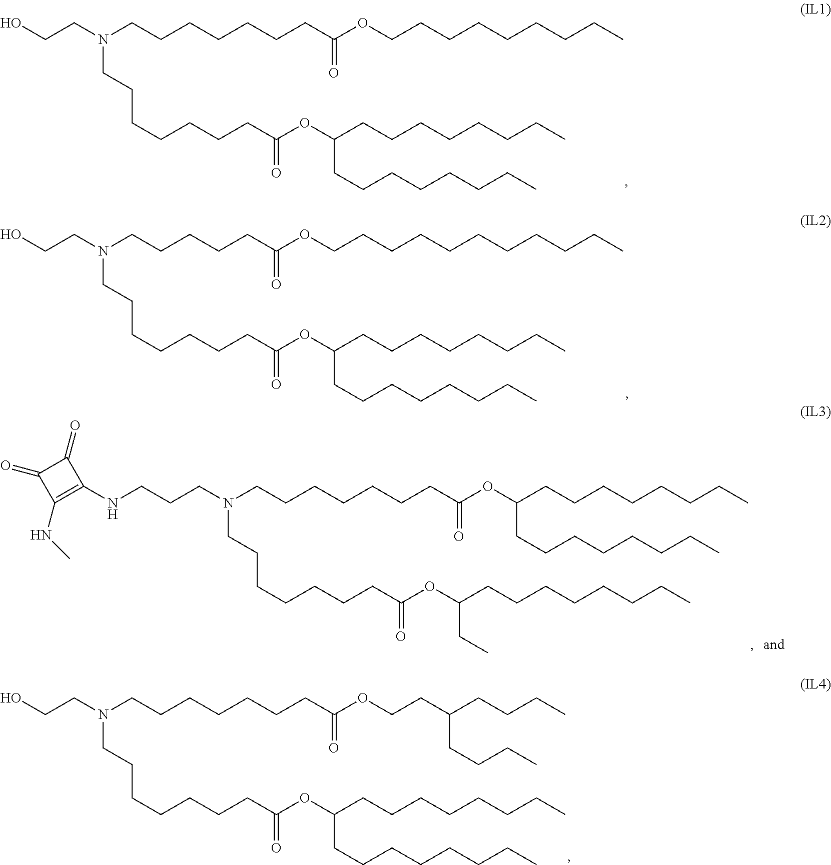

or an N-oxide or a salt thereof.

In some embodiments, the ionizable lipid is the compound:

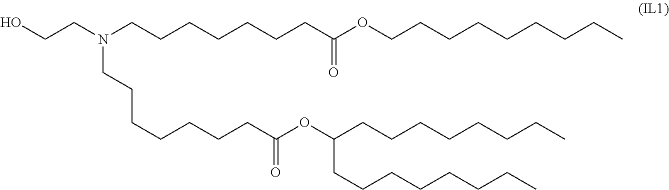 [SMILES: CCCCCCCCCOC(=O)CCCCCCCN(CCO)CCCCCCCC(=O)OC(CCCCCCCC)CCCCCCCC]

or an N-oxide or a salt thereof.

In some embodiments, the ionizable lipid is the compound:

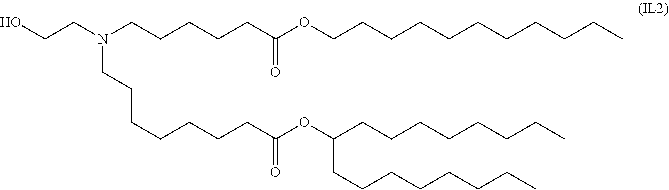 [SMILES: CCCCCCCCCCCOC(=O)CCCCCN(CCO)CCCCCCCC(=O)OC(CCCCCCCC)CCCCCCCC]

or an N-oxide or a salt thereof.

In some embodiments, the ionizable lipid is the compound:

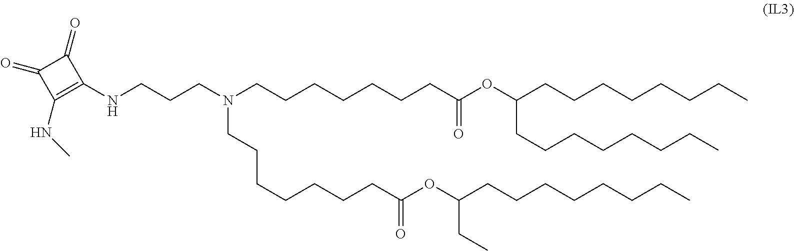 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or an N-oxide or a salt thereof.

In some embodiments, the ionizable lipid is the compound:

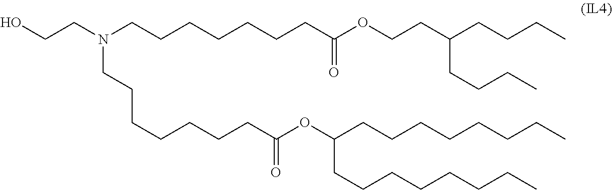 [SMILES: CCCCCCCCC(CCCCCCCC)OC(=O)CCCCCCCN(CCO)CCCCCCCC(=O)OCCC(CCCC)CCCC]

or an N-oxide or a salt thereof.

In some embodiments, the ionizable lipid is a compound of Formula (I):

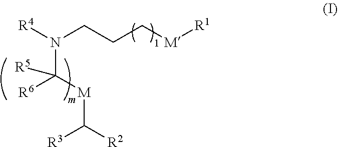

or an N-oxide or a salt thereof, wherein:
R¹ is:

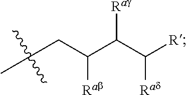

wherein

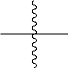

denotes a point of attachment;
R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and

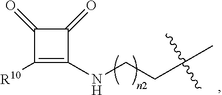

wherein

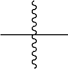

denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (I):

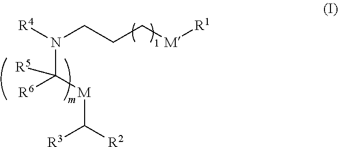

or an N-oxide or a salt thereof, wherein:
R¹ is:

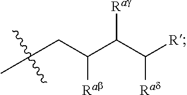

wherein

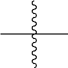

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is —(CH₂)ₙOH, wherein n is selected from 1, 2, 3, 4, and 5;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (I), or an N-oxide or a salt thereof, wherein:
R¹ is

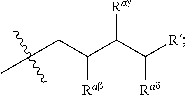

wherein

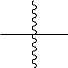

denotes a point of attachment;
R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is a compound of Formula (I), or an N-oxide or a salt thereof, wherein:
R¹ is

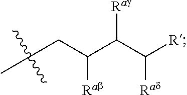

wherein

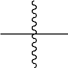

denotes a point of attachment;
R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 3; and
m is 7.

In some embodiments, the ionizable lipid is a compound of Formula (I), or an N-oxide or a salt thereof, wherein:
R¹ is

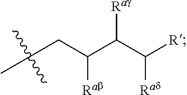

wherein

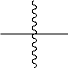

denotes a point of attachment;
R^{aβ} and R^{aδ} are each H;
R^{aγ} is C₂₋₁₂ alkyl;
R² and R³ are each C₁₋₁₄ alkyl;
R⁴ is —(CH₂)ₙOH;
n is 2;
each R⁵ is H;
wherein denotes a point of attachment;
each R⁶ is H;
M and M′ are each —C(O)O—;
R′ is C₁₋₁₂ alkyl;
l is 5; and
m is 7.

In some embodiments, the ionizable lipid is a compound of Formula (I):

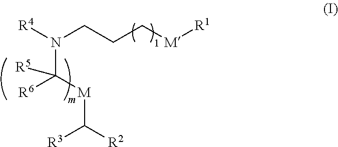

or an N-oxide or a salt thereof, wherein:
R¹ is:

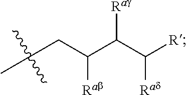

wherein

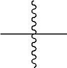

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is

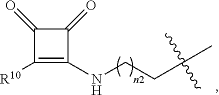

wherein

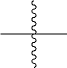

denotes a point of attachment;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments:
R¹ is

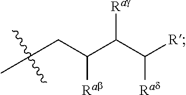

wherein

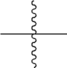

denotes a point of attachment;
R^{aβ}, R^{aγ}, and R^{aδ} are each H;
R^{aα} is C₂₋₁₂ alkyl;
R² and R³ are each C₁₋₁₄ alkyl;
R⁴ is

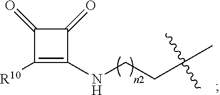

wherein

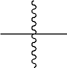

denotes a point of attachment;
wherein R¹⁰ is NH(C₁₋₆ alkyl);
wherein n2 is 2;
each R⁵ is H;
each R⁶ is H;
M and M′ are each —C(O)O—;
R′ is C₁₋₁₂ alkyl;
l is 5; and
m is 7.

In some embodiments, the ionizable lipid of Formula (I) is:

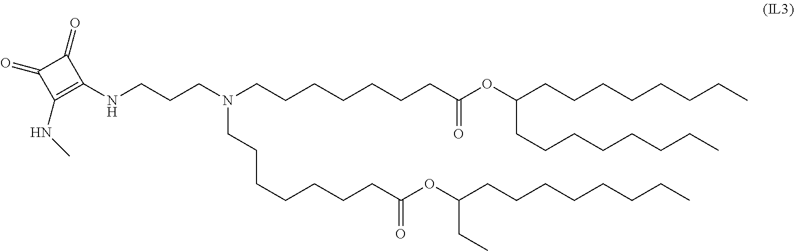 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or an N-oxide or a salt thereof.

In some embodiments, the ionizable lipid is a compound of Formula (II):

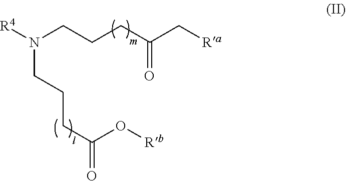

or an N-oxide or a salt thereof, wherein:
R′^{a} is R′^{branched} or R′^{cyclic}, wherein
   R′^{branched} is:

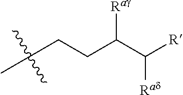

and R′^{cyclic} is:

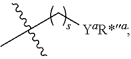

and
R′^{b} is:

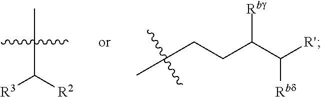

wherein

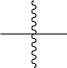

denotes a point of attachment;
R^{aγ} and R^{aδ} are each independently selected from H, C₁₋₁₂ alkyl, and C₂₋₁₂ alkenyl, wherein at least one of R^{aγ} and R^{aδ} is selected from C₁₋₁₂ alkyl and C₂₋₁₂ alkenyl;
R^{bγ} and R^{bδ} are each independently selected from H, C₁₋₁₂ alkyl, and C₂₋₁₂ alkenyl, wherein at least one of R^{bγ} and R^{bδ} is selected from C₁₋₁₂ alkyl and C₂₋₁₂ alkenyl;
R² and R³ are each independently selected from the C₁₋₁₄ alkyl and C₂₋₁₄ alkenyl;
R⁴ is selected from —(CH₂)ₙOH and

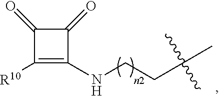

wherein

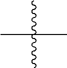

denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R′ independently is C₁₋₁₂ alkyl or C₂₋₁₂ alkenyl;
Y^{a} is a C₃₋₆ carbocycle;
R*″^{a} is selected from C₁₋₁₅ alkyl and C₂₋₁₅ alkenyl;
sis 2 or 3;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

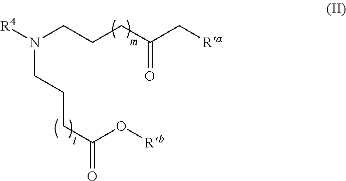

or an N-oxide or a salt thereof, wherein:
R′^{a} is R′^{branched} or R′^{cyclic}, wherein
R′^{branched} is:

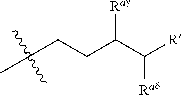

and R′^{b} is:

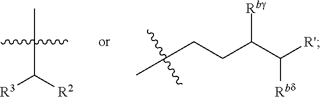

wherein [symbol] denotes a point of attachment;
R^{aγ} and R^{aδ} are each independently selected from H, C₁₋₁₂ alkyl, and C₂₋₁₂ alkenyl, wherein at least one of R^{aγ} and R^{aδ} is selected from C₁₋₁₂ alkyl and C₂₋₁₂ alkenyl;
R^{bγ} and R^{bδ} are each independently selected from H, C₁₋₁₂ alkyl, and C₂₋₁₂ alkenyl, wherein at least one of R^{bγ} and R^{bδ} is selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

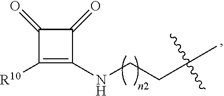

wherein [symbol] denotes a point of attachment;
wherein n is selected from of 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R′ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

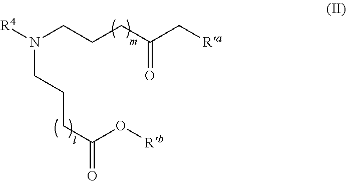

or an N-oxide or a salt thereof, wherein:
R′^{a} is R′^{branched} or R′^{cyclic}, wherein
R′^{branched} is:

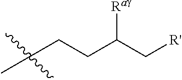

and R′^{b} is:

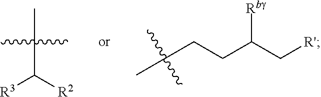

wherein [symbol] denotes a point of attachment;
R^{aγ} and R^{bγ} are each independently selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

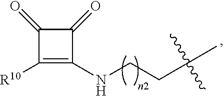

wherein [symbol] denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and
H; and wherein n2 is selected from 1, 2, 3, 4, 5, 6, 7, 8, 9, and 10;
each R′ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

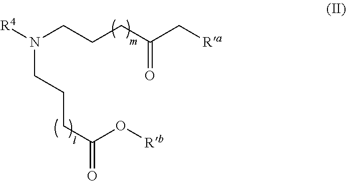

or an N-oxide or a salt thereof, wherein:
R′^{a} is R′^{branched} or R′^{cyclic};
R′^{branched} is

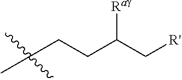

and R′^{b} is:

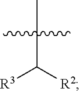

wherein [symbol] denotes a point of attachment;
R^{aγ} is selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

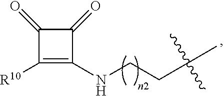

wherein [symbol] denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
R′ is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

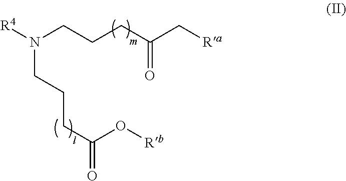

or an N-oxide or a salt thereof, wherein:
R′^{a} is R′^{branched} or R′^{cyclic};
R′^{branched} is

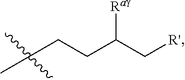

and R′^{b} is:

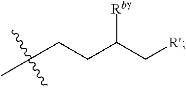

wherein [symbol] denotes a point of attachment;
R^{aγ} and R^{bγ} are each independently selected from C₁₋₁₂ alkyl and C₂₋₁₂ alkenyl;
R⁴ is selected from —(CH₂)ₙOH and

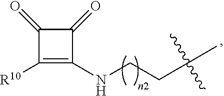

wherein [symbol] denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R′ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

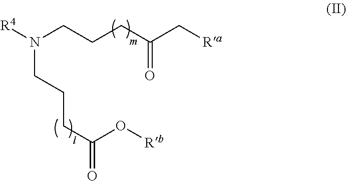

or an N-oxide or a salt thereof, wherein:
R′^{a} is R′^{branched} or R′^{cyclic}; wherein
R′^{branched} is:

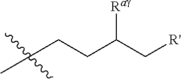

and R′^{b} is:

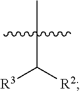

wherein [symbol] denotes a point of attachment;
R^{aγ} is selected from C₁₋₁₂ alkyl and C₂₋₁₂ alkenyl;
R² and R³ are each independently selected from C₁₋₁₄ alkyl and C₂₋₁₄ alkenyl;
R⁴ is —(CH₂)ₙOH wherein n is selected from 1, 2, 3, 4, and 5;
R′ is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, m and l are each independently selected from 4, 5, and 6. In some embodiments m and l are each 5.

In some embodiments each R′ independently is C₁₋₁₂ alkyl. In some embodiments, each R′ independently is C₂₋₅ alkyl.

In some embodiments, R′b is:

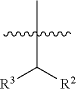

and R2 and R3 are each independently C1-14 alkyl.

In some embodiments, R′b is

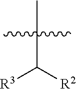

and R2 and R3 are each independently C₆₋₁₀ alkyl.

In some embodiments, R′b is:

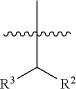

and R2 and R3 are each C8 alkyl.

In some embodiments, R′branched is:

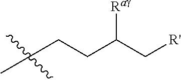

### and R′b is:

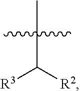

Raγ is C1-12 alkyl and R2 and R3 are each independently C₆₋₁₀ alkyl.

In some embodiments, R′branched is:

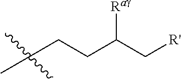

### and R′b is:

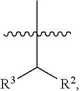

Raγ is a C2-6 alkyl and R2 and R3 are each independently C₆₋₁₀ alkyl. In some embodiments, R′branched is:

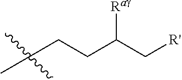

### and R′b is:

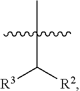

Raγ is C2-6 alkyl, and R2 and R3 are each a C8 alkyl.

In some embodiments, R′branched is:

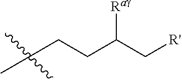

### and R′b is:

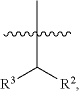

and Raγ and Rbγ are each C1-12 alkyl.

In some embodiments, R′branched is:

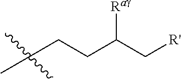

### R′b is:

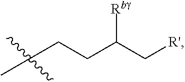

and Raγ and Rbγ are each a C2-6 alkyl.

In some embodiments, m and l are each independently selected from 4, 5, and 6 and each R′ independently is C1-12 alkyl. In some embodiments, m and l are each 5 and each R′ independently is C2-5 alkyl.

In some embodiments, R′branched is:

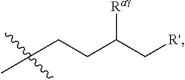

### R′b is:

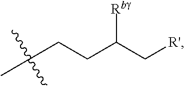

m and l are each independently selected from 4, 5, and 6, each R′ independently is C1-12 alkyl, and Raγ and Rbγ are each C1-12 alkyl.

In some embodiments, R′branched is:

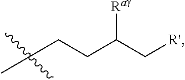

### R′b is:

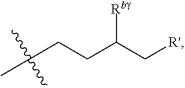

m and l are each 5, each R′ independently is a C2-5 alkyl, and Raγ and Rbγ are each a C2-6 alkyl.

In some embodiments, R′branched is:

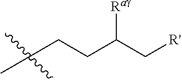

### and R′b is:

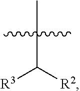

m and l are each independently selected from 4, 5, and 6, R′ is C1-12 alkyl, Raγ is C1-12 alkyl and R2 and R3 are each independently a C₆₋₁₀ alkyl.

In some embodiments, R′branched is:

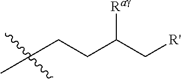

### and R′b is:

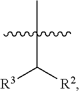

m and l are each 5, R′ is a C2-5 alkyl, Raγ is a C2-6 alkyl, and R2 and R3 are each a C8 alkyl.

In some embodiments, R⁴ is

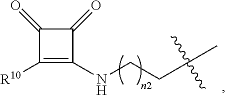

wherein R¹⁰ is NH(C₁₋₆ alkyl) and n2 is 2.

In some embodiments, R⁴ is

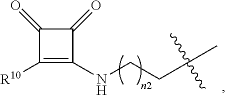

wherein R¹⁰ is NH(CH₃) and and n2 is 2.

In some embodiments, R′^{branched} is:

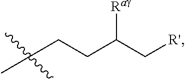

### R′b is:

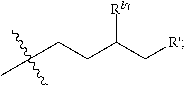

m and l are each independently selected from 4, 5, and 6; each R′ independently is C₁₋₁₂ alkyl; R^{aγ} and R^{bγ} are each C₁₋₁₂ alkyl; and R⁴ is

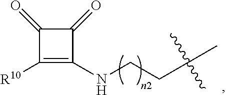

wherein R¹⁰ is NH(C₁₋₆ alkyl), and n2 is 2.

In some embodiments, R′^{branched} is:

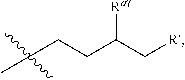

### R′b is:

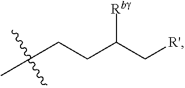

m and l are each 5, each R′ independently is a C₂₋₅ alkyl, R^{aγ} and R^{bγ} are each a C₂₋₆ alkyl, and R⁴ is

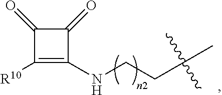

wherein R¹⁰ is NH(CH₃) and n2 is 2.

In some embodiments, R′^{branched} is:

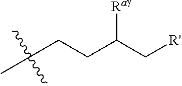

### and R′^{b} is:

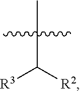

m and l are each independently selected from 4, 5, and 6, R′ is C₁₋₁₂ alkyl, R² and R³ are each independently a C₆₋₁₀ alkyl, R^{aγ} is C₁₋₁₂ alkyl, and R⁴ is

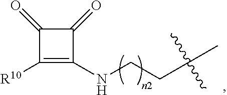

wherein R¹⁰ is NH(C₁₋₆ alkyl) and n2 is 2.

In some embodiments, R′^{branched} is:

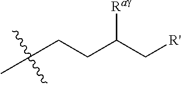

### and R′b is:

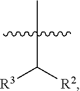

m and l are each 5, R′ is a C₂₋₅ alkyl, R^{aγ} is a C₂₋₆ alkyl, R² and R³ are each a C₈ alkyl, and R⁴ is

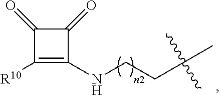

wherein R¹⁰ is NH(CH₃) and n2 is 2.

In some embodiments, R⁴ is —(CH₂)ₙOH and n is 2, 3, or 4. In some embodiments, R⁴ is —(CH₂)ₙOH and n is 2.

In some embodiments, R′^{branched} is:

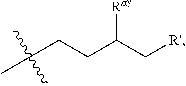

### R′^{b} is:

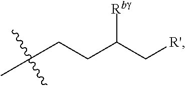

m and l are each independently selected from 4, 5, and 6, each R′ independently is C₁₋₁₂ alkyl, R^{aγ} and R^{bγ} are each C₁₋₁₂ alkyl, R⁴ is —(CH₂)ₙOH, and n is 2, 3, or 4.

In some embodiments, R′^{branched} is:

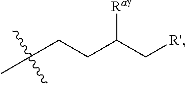

### R′^{b} is:

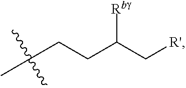

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable lipid is a compound of Formula (II):

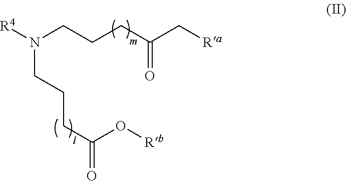

or an N-oxide or a salt thereof, wherein:
R′^{a} is R′^{branched} or R′^{cyclic}, wherein
R′^{branched} is:

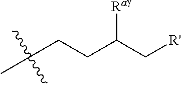

and R′^{b} is:

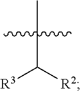

wherein [symbol] denotes a point of attachment;
R^{aγ} is C₁₋₁₂ alkyl;
R² and R³ are each independently C₁₋₁₄ alkyl;
R⁴ is —(CH₂)ₙOH wherein n is selected from 1, 2, 3, 4, and 5;
R′ is C₁₋₁₂ alkyl;
m is selected from 4, 5, and 6; and
l is selected from 4, 5, and 6.

In some embodiments, m and l are each 5, and n is 2, 3, or 4.

In some embodiments, R′ is a C₂₋₅ alkyl, R^{aγ} is a C₂₋₆ alkyl, and R² and R³ are each C₆₋₁₀ alkyl.

In some embodiments, m and l are each 5, n is 2, 3, or 4, R′ is a C₂₋₅ alkyl, R^{aγ} is C₂₋₆ alkyl, and R² and R³ are each a C₆₋₁₀ alkyl.

In some embodiments, the ionizable lipid is a compound of Formula (II-g):

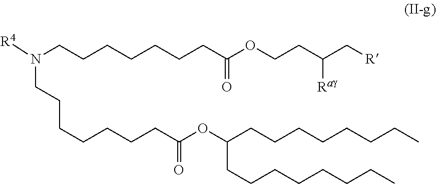

or an N-oxide or salt thereof, wherein:
R^{aγ} is C₂₋₆ alkyl;
R′ is C₂₋₅ alkyl; and
R⁴ is selected from —(CH₂)ₙOH and

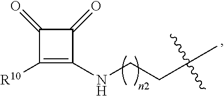

wherein [symbol] denotes a point of attachment,
wherein n is selected from 3, 4, and 5; and
wherein R¹⁰ is NH(C₁₋₆ alkyl); and
wherein n2 is selected from 1, 2, and 3.

In some embodiments, the ionizable lipid is a compound of Formula (II-h):

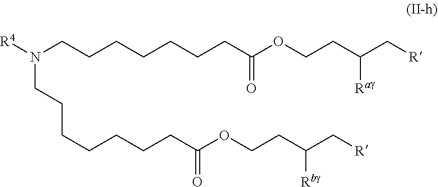

or an N-oxide or salt thereof, wherein:
R^{aγ} and R^{bγ} are each independently a C₂₋₆ alkyl;
each R′ independently is a C₂₋₅ alkyl; and
R⁴ is selected from —(CH₂)ₙOH and

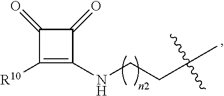

wherein [symbol] denotes a point of attachment,
wherein n is selected from 3, 4, and 5;
wherein R¹⁰ is NH(C₁₋₆ alkyl); and
wherein and n2 is selected from 1, 2, and 3.

In some embodiments, R⁴ is

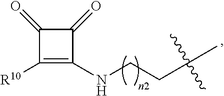

wherein R¹⁰ is NH(CH₃) and n2 is 2.

In some embodiments, R⁴ is —(CH₂)₂OH.

In some embodiments, the ionizable lipid is a compound having Formula (III):

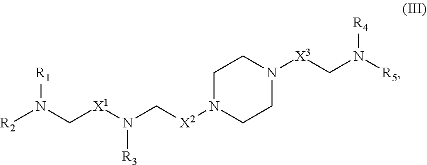

or an N-oxide or a salt thereof, wherein:
R₁, R₂, R₃, R₄, and R₅ are independently selected from C₅₋₂₀ alkyl, C₅₋₂₀ alkenyl, —R″MR′, —R*YR″, —YR″, and —R*OR″;
each M is independently selected
   from —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are each independently selected from a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from C₁₋₁₂ alkyl and C₂₋₁₂ alkenyl;
each R is independently selected from C₁₋₃ alkyl and a C₃₋₆ carbocycle;
   each R′ is independently selected from C₁₋₁₂ alkyl, C₂₋₁₂ alkenyl, and H; and
   each R″ is independently selected from C₃₋₁₂ alkyl and C₃₋₁₂ alkenyl, and wherein:
i) at least one of X¹, X², and X³ is not —CH₂—; and/or
ii) at least one of R₁, R₂, R₃, R₄, and R₅ is —R″MR′.

In some embodiments, R₁, R₂, R₃, R₄, and R₅ are each C₅₋₂₀ alkyl; X¹ is —CH₂—; and X² and X³ are each —C(O)—.

In some embodiments, the compound of Formula (III) is:

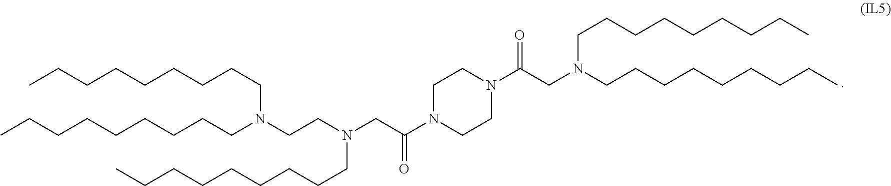 [SMILES: CCCCCCCCCN(CCCCCCCCC)CCN(CCCCCCCCC)CC(=O)N1CCN(C(=O)CN(CCCCCCCCC)CCCCCCCCC)CC1]

### Phospholipid

Phospholipids, as defined herein, are any lipids that comprise a phosphate group. Phospholipids are a subset of non-cationic lipids. The LNP core may include one or more phospholipids, such as one or more (poly)unsaturated lipids. Phospholipids may assemble into one or more lipid bilayers. In general, phospholipids may include a phospholipid moiety and one or more fatty acid moieties. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

In some embodiments, the LNP comprises about 5 mol % to about 15 mol %, about 8 mol % to about 13 mol %, or about 10 mol % to about 12 mol % of phospholipid.

In some embodiments, the phospholipid is a compound of Formula (IV):

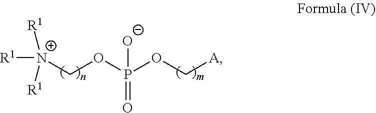

or a salt thereof, wherein:
each R¹ is independently H or optionally substituted alkyl; or optionally two R¹ are joined together with the intervening atoms to form optionally substituted monocyclic cycloalkyl or optionally substituted monocyclic heterocyclyl; or optionally three R¹ are joined together with the intervening atoms to form optionally substituted bicyclic cycloalkyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

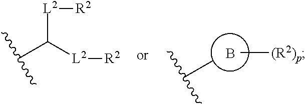

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2;
provided that the compound is not of the formula:

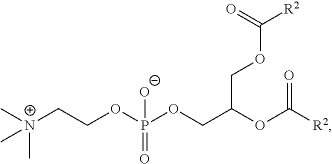

wherein each instance of R² is independently unsubstituted alkyl, unsubstituted alkenyl, or unsubstituted alkynyl.

In some embodiments, the phospholipids is selected from:
1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-diphytanoyl-sn-glycero-3-phosphoethanolamine (ME 16.0 PE),
1,2-diphytanoyl-sn-glycero-3-phosphocholine (4ME 16:0 PC),
1,2-diphytanoyl-sn-glycero-3-phospho-(1′-rac-glycerol) (sodium salt) (4ME 16:0 PG),
1,2-diphytanoyl-sn-glycero-3-phospho-L-serine (sodium salt) (4ME 16:0 PS),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), and Sphingomyelin.

In some embodiments, the phospholipid is DSPC, DOPE, or combinations thereof. In some embodiments, the phospholipid is DSPC. In some embodiments, the phospholipid is DOPE. In some embodiments, the phospholipid is 4ME 16:0 PE, 4ME 16:0 PC, 4ME 16:0 PG, 4ME 16:0 PS, or combination thereof.

In some embodiments, the phospholipid is N-lauroyl-D-erythro-sphinganylphosphorylcholine.

### Alternative Lipids

In certain embodiments, an alternative lipid is used in place of a phospholipid of the invention. Non-limiting examples of such alternative lipids include the following:

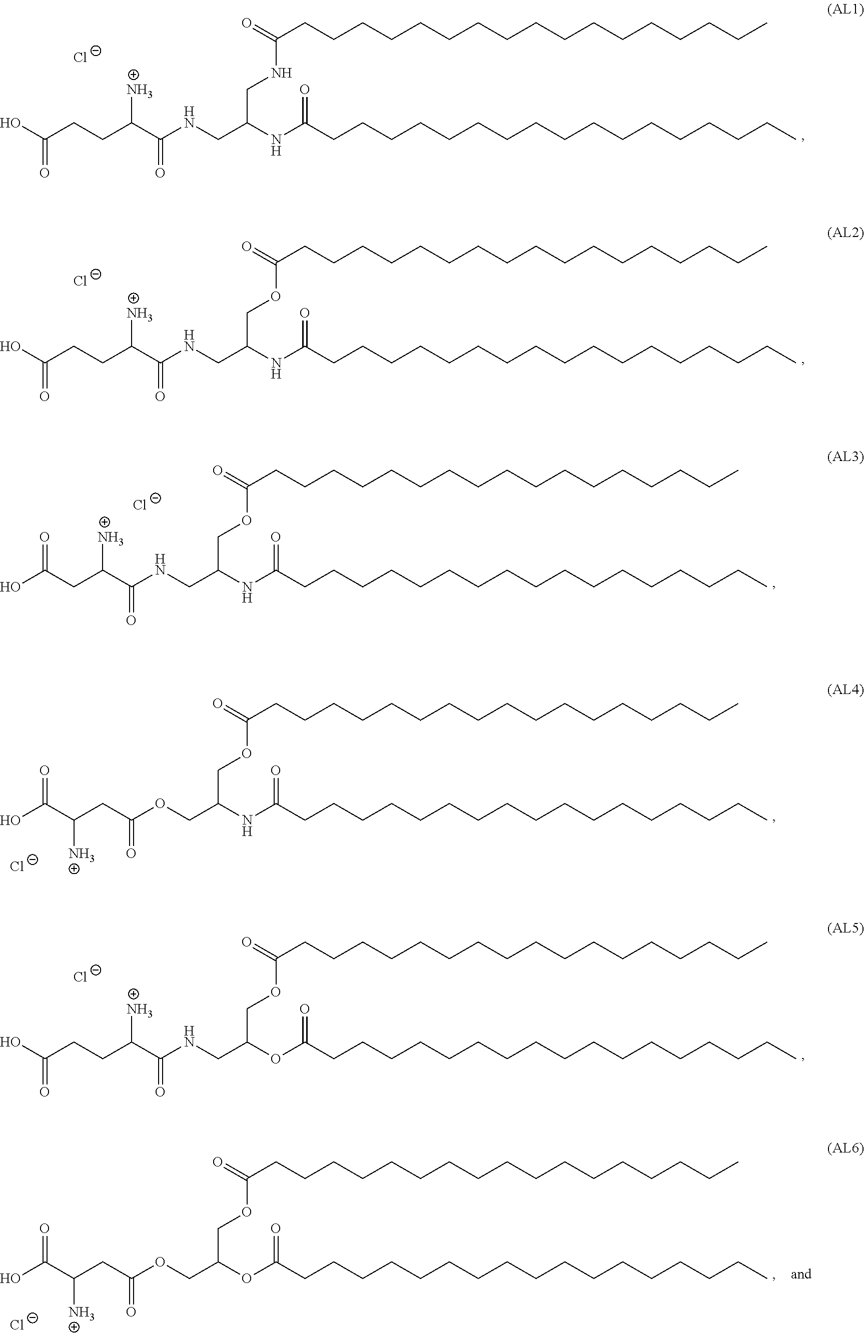

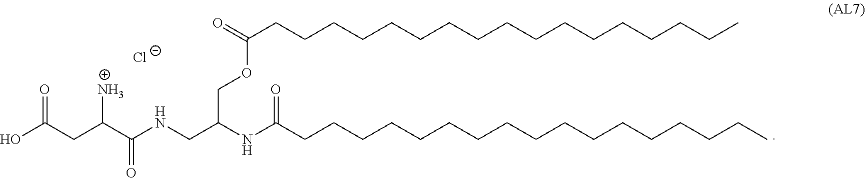 [SMILES: CCCCCCCCCCCCCCCCCC(=O)NC(CNC(=O)C([NH3+])CC(=O)O)COC(=O)CCCCCCCCCCCCCCCCC ; [Cl-]]

### Structural Lipid

The LNP core may include one or more structural lipids. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols.

In some embodiments, the structural lipid is alpha-tocopherol. In certain embodiments, the structural lipid is a steroid. In certain embodiments, the structural lipid is cholesterol. In certain embodiments, the structural lipid is an analog of cholesterol. In some embodiments, the structural lipid is β-sitosterol. In certain embodiments, the structural lipid is cholesteryl hemisuccinate. Cholesteryl hemisuccinate has the following structure:

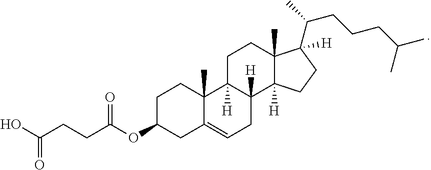

In some embodiments, the LNP comprises about 20 mol % to about 60 mol %, about 30 mol % to about 50 mol %, or about 35 mol % to about 40 mol % of structural lipid. In some embodiments, the LNP comprises about 35 mol % of structural lipid. In some embodiments, the LNP comprises about 40 mol % structural lipid.

### PEG and PEG-Modified Lipids

The LNP core may include one or more molecules comprising polyethylene glycol (PEG), such PEG-modified lipids. Such species may be alternately referred to as PEGylated lipids. The PEG-lipid is a lipid modified with polyethylene glycol. The PEG-lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof.

In some embodiments, the PEG lipid is a compound of Formula (V):

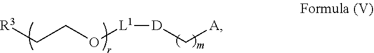

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted cycloalkylene, optionally substituted heterocyclylene, optionally substituted arylene, optionally substituted heteroarylene, —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
D is a moiety obtained by click chemistry or a moiety cleavable under physiological conditions;
m is 0, 1, 2, 3, 4, 5, 6, 7, 8, 9, or 10;
A is of the formula:

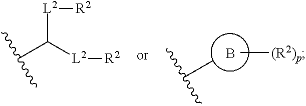

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2.

In some embodiments, the PEG lipid is PEG-c-DOMG, PEG-DMG, PEG-DLPE, PEG-DMPE, PEG-DPPC, or PEG-DSPE. In some embodiments, the PEG lipid is PEG-DMG. In some embodiments, the PEG lipid is PEG-DMG 2 k. In some embodiments, a PEG lipid has the structure:

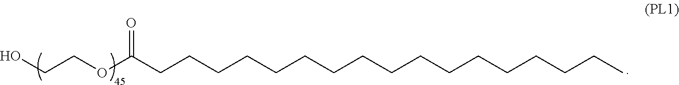

DMG-PEG 2 k has the following structure:

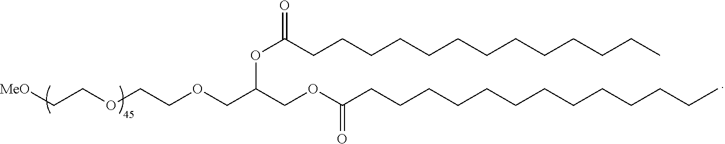

In some embodiments, the PEG-modified lipid is a modified form of PEG-DMG.

In one embodiment, PEG-lipids useful in the present invention can be PEGylated lipids described in International Publication No. WO2012099755, the contents of which is herein incorporated by reference in its entirety.

Any of the PEG-lipids described herein may be modified to comprise one or more hydroxyl group on the PEG chain (OH-PEG-lipids) or one or more hydroxyl group on the lipid (PEG-lipid-OH). In some embodiments, the PEG-lipid is an OH-PEG-lipid. In some embodiments, the OH-PEG-lipid comprises a hydroxyl group at the terminus of the PEG chain. In some embodiments, the PEG-lipids described herein may be modified to comprise one or more alkyl group on the PEG chain (alkyl-PEG-lipid). In some embodiments, the alkyl-PEG-lipid is a methoxy-PEG-lipid.

In some embodiments, the LNP comprises about 0.1 mol % to about 5.0 mol %, about 0.5 mol % to about 5.0 mol %, about 1.0 mol % to about 5.0 mol %, about 1.0 mol % to about 2.5 mol %, about 0.5 mol % to about 2.0 mol %, or about 1.0 mol % to about 1.5 mol % of PEG-lipid. In some embodiments, the LNP comprises about 1.5 mol % or about 3.0 mol % PEG-lipid.

Certain of the LNPs provided herein comprise no or low levels of PEG-lipid. Some LNPs comprise less than 0.5 mol % PEG-lipid.

In some embodiments, PEG is used as a stabilizer. In some embodiments, the PEG stabilizer is a PEG-lipid. In some embodiments, the LNP comprises less than 0.5 mol % PEG stabilizer.

### Payload Molecules

The lipid nanoparticle compositions of the disclosure can be used to deliver a wide variety of different payloads to cells. The payload can be a therapeutic or prophylactic agent capable of mediating (e.g., directly mediating or via a bystander effect) a therapeutic or prophylactic effect in such cell. Typically the payload delivered by the composition is a nucleic acid, although non-nucleic acid agents, such as small molecules, chemotherapy drugs, peptides, polypeptides and other biological molecules are also encompassed by the disclosure. Nucleic acids that can be delivered include DNA-based molecules (i.e., comprising deoxyribonucleotides) and RNA-based molecules (i.e., comprising ribonucleotides). Furthermore, the nucleic acid can be a naturally occurring form of the molecule or a chemically-modified form of the molecule (e.g., comprising one or more modified nucleotides).

In one embodiment, the therapeutic or prophylactic is an agent that enhances (i.e., increases, stimulates, upregulates) protein expression. Non-limiting examples of types of therapeutic or prophylactic agents that can be used for enhancing protein expression include RNAs, mRNAs, dsRNAs, CRISPR/Cas9 technology, ssDNAs and DNAs (e.g., expression vectors).

In one embodiment, the therapeutic or prophylactic agent is an agent that reduces (i.e., decreases, inhibits, downregulates) protein expression. Non-limiting examples of types of therapeutic or prophylactic that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

In one embodiment, the therapeutic or prophylactic is a peptide therapeutic agent. In one embodiment the therapeutic or prophylactic a polypeptide therapeutic agent. In some embodiments, the therapeutic or prophylactic agent comprises an mRNA encoding: a secreted protein; a membrane-bound protein; or an intercellular protein, or peptides, polypeptides or biologically active fragments thereof.

In some embodiments, at least about 50%, at least about 75%, at least about 90%, or at least about 95% of the payload is encapsulated within the lipid nanoparticle. In some embodiments, about 50% to about 99%, about 65% to about 99%, about 75% to about 95%, or about 80% to about 95% of the payload is encapsulated within the lipid nanoparticle.

### Cells

The LNPs of the invention can be used to deliver payload molecules to a population of cells. In some embodiments, the LNPs are contacted with a population of cells. In some embodiments, about 10% or greater, 15% or greater, 20% or greater, or 30% or greater of the cell population has accumulated the LNPs when the LNPs are contacted with the cell population. In some embodiments, about 1% to about 75%, about 5% to about 50%, about 10% to about 40%, or about 15% to about 25% of the cell population has accumulated the LNPs when the LNPs are contacted with the cell population.

In some embodiments, about 5% or greater, about 10% or greater, or about 20% or greater of cells in the population of cells expresses the payload when the LNP is contacted with the population of cells. In some embodiments, about 0.5% to about 50%, about 1% to about 40%, about 3% to about 20%, or about 5% to about 15% of cells in the population of cells expresses the payload when the LNP is contacted with the population of cells.

In some embodiments, the cell population is an epithelial cell population. In some embodiments, the cell population is a respiratory epithelial cell population. In some embodiments, the respiratory epithelial cell population is a lung cell population. In some embodiments, the respiratory epithelial cell population is a nasal cell population. In some embodiments, the respiratory epithelial cell population is an alveolar epithelial cell population. In some embodiments, the respiratory epithelial cell population is a bronchial epithelial cell population. In some embodiments, the respiratory epithelial cell population is an HBE population. In some embodiments, the cell population is a HeLa population.

### Pharmaceutical Compositions and Formulations

The present disclosure provides pharmaceutical compositions and formulations that comprise any of LNPs described herein.

Pharmaceutical compositions or formulations can optionally comprise one or more additional active substances, e.g., therapeutically and/or prophylactically active substances. Pharmaceutical compositions or formulations of the present disclosure can be sterile and/or pyrogen-free. General considerations in the formulation and/or manufacture of pharmaceutical agents can be found, for example, in Remington: The Science and Practice of Pharmacy 21^{st} ed., Lippincott Williams & Wilkins, 2005 (incorporated herein by reference in its entirety). In some embodiments, compositions are administered to humans, human patients or subjects. For the purposes of the present disclosure, the phrase “active ingredient” generally refers to the nanoparticle comprising the polynucleotides or polypeptide payload to be delivered as described herein.

Formulations and pharmaceutical compositions described herein can be prepared by any method known or hereafter developed in the art of pharmacology. In general, such preparatory methods include the step of associating the nanoparticle with an excipient and/or one or more other accessory ingredients, and then, if necessary and/or desirable, dividing, shaping and/or packaging the product into a desired single- or multi-dose unit.

A pharmaceutical composition or formulation in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered.

Although the descriptions of pharmaceutical compositions and formulations provided herein are principally directed to pharmaceutical compositions and formulations that are suitable for administration to humans, it will be understood by the skilled artisan that such compositions are generally suitable for administration to any other animal, e.g., to non-human animals, e.g. non-human mammals.

A pharmaceutically acceptable excipient, as used herein, includes, but is not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cryoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (povidone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC® F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation is a potential degradation pathway for mRNA, especially for liquid mRNA formulations. In order to prevent oxidation, antioxidants can be added to the formulations. Exemplary antioxidants include, but are not limited to, alpha tocopherol, ascorbic acid, acorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions are maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition described here can contain a bulking agent in lyophilized polynucleotide formulations to yield a “pharmaceutically elegant” cake, stabilize the lyophilized polynucleotides during long term (e.g., 36 month) storage. Exemplary bulking agents of the present disclosure can include, but are not limited to sucrose, trehalose, mannitol, glycine, lactose, raffinose, and combinations thereof.

The compositions can be in a liquid form or a solid form. In some embodiments, the compositions or formulations are in a liquid form. In some embodiments, the compositions are suitable for inhalation. The compositions can be administered to the pulmonary tract.

Aerosolized pharmaceutical formulations can be delivered to the lungs, preferably using a number of commercially available devices.

Compositions can be administered to the respiratory tract by suitable methods such as intranasal instillation, intratracheal instillation, and intratracheal injection. In some embodiments, the compositions or the nanoparticle is administered by intranasal, intrabronchial, or pulmonary administration. For example, the compositions and nanoparticles are administered by nebulizer or inhaler.

In some embodiments, the compositions are delivered into the lungs by inhalation of an aerosolized pharmaceutical formulation. Inhalation can occur through the nose and/or the mouth of the subject. Administration can occur by self-administration of the formulation while inhaling, or by administration of the formulation via a respirator to a subject on a respirator. Exemplary devices for delivering formulations to the lung include, but are not limited to, dry powder inhalers, pressurized metered dose inhalers, nebulizers, and electrohydrodynamic aerosol devices.

Liquid formulations can be administered to the lungs of a patient using a pressurized metered dose inhaler (pMDI). pMDIs generally include at least two components: a canister in which the liquid formulation is held under pressure in combination with one or more propellants, and a receptacle used to hold and actuate the canister. The canister may contain a single or multiple doses of the formulation. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the liquid formulation is atomized, forming an aerosol. pMDIs typically employ one or more propellants to pressurize the contents of the canister and to propel the liquid formulation out of the receptacle outlet, forming an aerosol. Any suitable propellants may be utilized. The propellant may take a variety of forms. For example, the propellant may be a compressed gas or a liquefied gas.

The liquid formulations can also be administered using a nebulizer. Nebulizers are liquid aerosol generators that convert the liquid formulation into mists or clouds of small droplets, preferably having diameters less than 5 microns mass median aerodynamic diameter, which can be inhaled into the lower respiratory tract. This process is called atomization. The droplets carry the one or more active agents into the nose, upper airways or deep lungs when the aerosol cloud is inhaled. Any type of nebulizer may be used to administer the formulation to a patient, including, but not limited to pneumatic (jet) nebulizers and electromechanical nebulizers. Pneumatic (jet) nebulizers use a pressurized gas supply as a driving force for atomization of the liquid formulation. Compressed gas is delivered through a nozzle or jet to create a low pressure field which entrains a surrounding liquid formulation and shears it into a thin film or filaments. The film or filaments are unstable and break up into small droplets that are carried by the compressed gas flow into the inspiratory breath. Baffles inserted into the droplet plume screen out the larger droplets and return them to the bulk liquid reservoir. Electromechanical nebulizers use electrically generated mechanical force to atomize liquid formulations. The electromechanical driving force can be applied, for example, by vibrating the liquid formulation at ultrasonic frequencies, or by forcing the bulk liquid through small holes in a thin film. The forces generate thin liquid films or filament streams which break up into small droplets to form a slow moving aerosol stream which can be entrained in an inspiratory flow. Liquid formulations can also be administered using an electrohydrodynamic (EHD) aerosol device. EHD aerosol devices use electrical energy to aerosolize liquid drug solutions or suspensions.

Dry powder inhalers (DPIs) typically use a mechanism such as a burst of gas to create a cloud of dry powder inside a container, which can then be inhaled by the subject. In a DPI, the dose to be administered is stored in the form of a non-pressurized dry powder and, on actuation of the inhaler, the particles of the powder are inhaled by the subject. In some cases, a compressed gas (i.e., propellant) may be used to dispense the powder, similar to pressurized metered dose inhalers (pMDIs). In some cases, the DPI may be breath actuated, meaning that an aerosol is created in precise response to inspiration. Typically, dry powder inhalers administer a dose of less than a few tens of milligrams per inhalation to avoid provocation of cough. Examples of DPIs include the Turbohaler® inhaler (Astrazeneca, Wilmington, Del.), the Clickhaler® inhaler (Innovata, Ruddington, Nottingham, UKL), the Diskus® inhaler (Glaxo, Greenford, Middlesex, UK), the Easy Haler® (Orion, Expoo, FI), the Exubera® inhaler (Pfizer, New York, N.Y.), the Qdose® inhaler (Microdose, Monmouth Junction, N.J.), and the Spiros® inhaler (Dura, San Diego, Calif.).

The pharmaceutical compositions of the invention are administered in an effective amount to cause a desired biological effect, e.g., a therapeutic or prophylactic effect, e.g., owing to expression of a normal gene product to supplement or replace a defective protein or to reduce expression of an undesired protein, as measured by, in some embodiments, the alleviation of one or more symptoms. The formulations may be administered in an effective amount to deliver LNP to, e.g., the apical membrane of respiratory and non-respiratory epithelial cells to deliver a payload.

### Methods of Use

The lipid amine compounds of the invention can be used to prepare lipid nanoparticle compositions which can be loaded with a payload and administered to cells, such as cells in patients for the treatment of disease. Accordingly, the present invention includes methods of delivering a payload into a cell such as by contacting the cell with a lipid nanoparticle composition disclosed herein.

In some embodiments, the cell is an epithelial cell. In some embodiments, the cell is an airway epithelial cell. In some embodiments, the cell is a respiratory epithelial cell. The respiratory epithelial cell can be, for example, a lung cell, a nasal cell, an alveolar epithelial cell, or a bronchial epithelial cell. In some embodiments, the cell is an HBE cell or a HeLa population. In some embodiments, the cell is in a patient.

In some embodiments, the payload is a polynucleotide or polypeptide. The polynucleotide include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof. In some embodiments, the polynucleotide is mRNA, rRNA, or tRNA. In some embodiments, the polynucleotide is mRNA.

The lipid nanoparticle composition can be administered to patient by intranasal, intrabronchial, or pulmonary administration. For example, the compositions and nanoparticles can be administered by nebulizer or inhaler.

As will be appreciated by those skilled in the art, the lipid amines disclosed herein have additional uses. For example, lipid amines can be used to treat inflammatory diseases. Lipid amines can also be used as antimicrobial agents.

### Kits and Devices

The present disclosure provides a variety of kits for conveniently and/or effectively using the claimed nanoparticles of the present disclosure. Typically kits will comprise sufficient amounts and/or numbers of components to allow a user to perform multiple treatments of a subject(s) and/or to perform multiple experiments.

In one aspect, the present disclosure provides kits comprising the nanoparticles of the present disclosure.

The kit can further comprise packaging and instructions and/or a delivery agent to form a formulation composition. The delivery agent can comprise a saline, a buffered solution, a lipidoid or any delivery agent disclosed herein. In one embodiment, such a kit further comprises an administration device such as a nebulizer or an inhaler.

### Process of Preparing LNPs

The present disclosure also provides a process of preparing a lipid nanoparticle composition comprising combining the lipid amine compound disclosed herein, or a salt thereof, with one or more additional lipids selected from:
(i) an ionizable lipid,
(ii) a phospholipid,
(iii) a structural lipid, and
(iv) a PEG-lipid.

In some embodiments, the combining comprises nanoprecipitation. Nanoprecipitation is the unit operation in which the nanoparticles are self-assembled from their individual lipid components by way of kinetic mixing and subsequent maturation and continuous dilution.

In some embodiments, the present disclosure provides a process for preparing a lipid nanoparticle composition comprising:
(1) mixing of an aqueous input and an organic input,
(2) optionally allowing for maturation of the resulting lipid nanoparticle composition, and
(3) optionally diluting the resulting lipid nanoparticle composition.

In some embodiments, the process includes the continuous inline combination of more than 1 (e.g., three) liquid streams with one inline maturation step.

In some embodiments, the organic input comprises a lipid amine compound disclosed herein (e.g., Formula A1) and one or more additional lipids. In some embodiments, the organic input comprises a lipid amine compound disclosed herein, an ionizable lipid, a phospholipid, a structural lipid, and optionally a PEG-lipid. In some embodiments, the organic input comprises a lipid amine compound disclosed herein, an ionizable lipid, a phospholipid, and a structural lipid.

In some embodiments, the organic input comprises a lipid amine and one or more additional lipids dissolved in an organic solvent. In some embodiments, the organic solvent is dimethylsulfoxide, acetone, acetonitrile, ethylene glycol, 1,4-dioxane, 1,3-butanediol, 2-butoxyethanol, or dimethylformamide. In some embodiments, the organic solvent is an organic alcohol. In some embodiments, the organic alcohol is a C₁₋₁₀ hydroxyalkyl. In some embodiments, the organic alcohol is methanol, ethanol, or isopropanol. In some embodiments, the organic alcohol is ethanol.

In some embodiments, the organic input has a lipid concentration of about 1 to about 50 mM, about 5 to about 35 mM, about 10 to about 20 mM, or about 12.5 mM.

In some embodiments, the organic input comprises about 20 mol % to about 50 mol %, about 25 mol % to about 45 mol %, or about 30 mol % to about 40 mol % of ionizable lipid with respect to total lipids. In some embodiments, the organic input comprises about 5 mol % to about 20 mol %, about 8 mol % to about 15 mol %, or about 10 mol % to about 12 mol % of phospholipid with respect to total lipids. In some embodiments, the organic input comprises about 30 mol % to about 50 mol %, about 35 mol % to about 45 mol %, or about 37 mol % to about 42 mol % of structural lipid with respect to total lipids. In some embodiments, the organic input comprises about 0.1 mol % to about 5 mol %, about 0.5 mol % to about 2.5 mol %, or about 1 mol % to about 2 mol % of PEG-lipid with respect to total lipids. In some embodiments, the organic input comprises about 5 mol % to about 30 mol %, about 10 mol % to about 25 mol %, or about 12 mol % to about 20 mol % of lipid amine with respect to total lipids.

In some embodiments, the lipid solution comprises:
about 30 mol % to about 40 mol % of ionizable lipid;
about 10 mol % to about 12 mol % of phospholipid;
about 37 mol % to about 42 mol % of structural lipid;
about 1 mol % to about 2 mol % of PEG-lipid; and
about 12 mol % to about 20 mol % of lipid amine; each with respect to total lipids.

In some embodiments, the lipid solution comprises:
about 33 mol % of ionizable lipid;
about 11 mol % to about 12 mol % of phospholipid;
about 39.5 mol % of structural lipid;
about 1.5 mol % of PEG-lipid; and
about 15 mol % lipid amine; each with respect to total lipids.

In some embodiments, the aqueous input comprises water. In some embodiments, the aqueous input comprises an aqueous buffer solution. In some embodiments, the aqueous buffer solution has a pH of about 3.5 to about 4.5. In further embodiments, the aqueous buffer solution has a pH of about 4. In some embodiments, the aqueous buffer solution has a pH of about 4.6 to about 6.5. In some embodiments, the aqueous buffer solution has a pH of about 5. In some embodiments, the aqueous buffer solution can comprise an acetate buffer, a citrate buffer, a phosphate buffer, or a Tris buffer. In some embodiments, the aqueous buffer solution comprises an acetate buffer or a citrate buffer. In further embodiments, the aqueous buffer solution is an acetate buffer, such as a sodium acetate buffer. In some embodiments, the aqueous buffer solution has a buffer concentration greater than about 30 mM. In some embodiments, the aqueous buffer solution has a buffer concentration greater than about 40 mM. In some embodiments, the aqueous buffer solution has a buffer concentration of about 30 mM to about 100 mM. In some embodiments, the aqueous buffer solution has a buffer concentration of about 40 mM to about 75 mM. In some embodiments, the aqueous buffer solution has a buffer concentration of about 25 mM.

In some embodiments, the aqueous input further comprises a payload. In some embodiments, the payload is a nucleic acid such as RNA or DNA. In some embodiments, the RNA is mRNA. In some embodiments, the aqueous input can include the nucleic acid at a concentration of about 0.05 to about 5.0 mg/mL, 0.05 to about 2.0 mg/mL, about 0.05 to about 1.0 mg/mL, about 0.1 to about 0.5 mg/mL, or about 0.2 to about 0.3 mg/mL. In some embodiments, the nucleic acid concentration is about 0.25 mg/mL.

The mixing of the aqueous and organic inputs can occur in a scale-appropriate mixer, which is designed to allow continuous, high-energy, combination of the aqueous input with the organic input. In some embodiments, the aqueous input and organic input flow simultaneously into the mixing hardware continuously throughout this operation. In some embodiments, the aqueous input and organic input are mixed at a volume ratio of about 5:1, about 4:1, about 3:1, about 2:1, about 1:1, about 1:2, about 1:3, about 1:4, or about 1:4 aqueous input to organic input. The precipitation of the lipid amine and one or more additional lipids can be caused by reducing the organic solvent content.

In some embodiments, the maturation comprises controlled residence time. In some embodiments, the residence time is about 5 to about 120 seconds, about 10 to about 90 seconds, about 20 to about 70 seconds, about 30 to about 60 seconds, about 30 seconds, about 45 seconds, or about 60 seconds.

In some embodiments, the nanoparticles are diluted with a dilution buffer. The dilution buffer can be an aqueous buffer solution with a buffer concentration of about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM. In some embodiments, the buffer concentration is about 30 mM to about 75 mM, about 30 mM to about 60 mM, or about 30 mM to about 50 mM. In some embodiments, the dilution buffer comprises an acetate buffer, a citrate buffer, a phosphate buffer, or a tris buffer. In some embodiments, the dilution buffer comprises an acetate buffer or a citrate buffer. In further embodiments, the dilution buffer is an acetate buffer, such as a sodium acetate. In some embodiments, the pH of the dilution buffer is about 3 to about 7, about 3 to about 6, about 3 to about 5, about 4, about 5, about 5.5, or about 6. In some embodiments, the dilution buffer comprises the same buffer as in the aqueous input.

In some embodiments, the process of preparing a lipid nanoparticle composition further comprises filtering. In some embodiments, the filtering comprises dialysis. In some embodiments, the dialysis is performed using a Slide-A-Lyzer dialysis cassette. In some embodiments, the dialysis cassette has a molecular weight cut off of about 5 kDa, about 10 kDa, about 15 kDa, or about 20 kDa. The dialysis can be carried out at about 25° C., about 20° C., about 10° C., about 5° C., or about 4° C. In some embodiments, the filtering further comprises filtering through a 0.1 μm to about 1 μm filter. In some embodiments, the filtering further comprises filtering through a 0.22 μm filter.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the schemes below.

The reactions for preparing compounds described herein can be carried out in suitable solvents, which can be readily selected by one of skill in the art of organic synthesis. Suitable solvents can be substantially non-reactive with the starting materials (reactants), the intermediates, or products at the temperatures at which the reactions are carried out, (e.g., temperatures, which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

Compounds of Formula A1 can be prepared, e.g., using a process as illustrated in the schemes below:

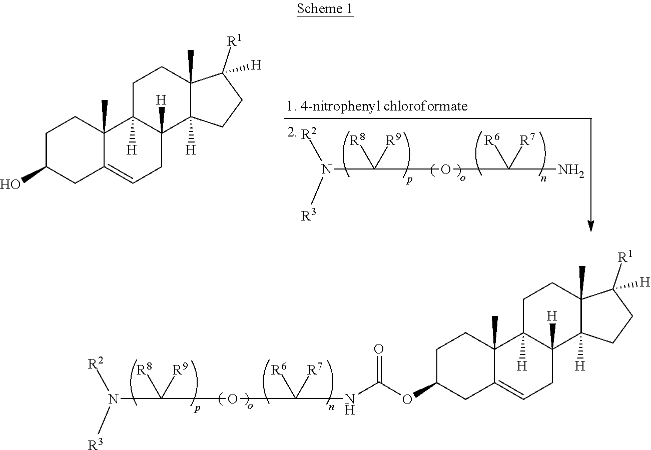

Compounds of Formula A1 can be prepared via the synthetic route outlined in Scheme 1. An appropriate reaction between cholesterol or a cholesterol derivative (such as stigmasterol) and 4-nitrophenyl chloroformate can be carried out under suitable conditions (such as using triethylamine and 4-dimethylaminopyridine). The product of said reaction can be reacted with an amine under suitable conditions (such as using triethylamine) to generate a precursor to a compound of Formula A1 or a compound of Formula A1.

### Definitions

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the present disclosure. Where a value is explicitly recited, it is to be understood that values which are about the same quantity or amount as the recited value are also within the scope of the present disclosure. Where a combination is disclosed, each subcombination of the elements of that combination is also specifically disclosed and is within the scope of the present disclosure. Conversely, where different elements or groups of elements are individually disclosed, combinations thereof are also disclosed. Where any element of an present disclosure is disclosed as having a plurality of alternatives, examples of that present disclosure in which each alternative is excluded singly or in any combination with the other alternatives are also hereby disclosed; more than one element of an present disclosure can have such exclusions, and all combinations of elements having such exclusions are hereby disclosed.

The term “about” as used in connection with a numerical value throughout the specification and the claims denotes an interval of accuracy, familiar and acceptable to a person skilled in the art. Such interval of accuracy is, for example ±10%.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereometric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. For example, contacting a cell (e.g., a mammalian cell) with a nanoparticle composition means that the cell and a nanoparticle are made to share a physical connection. Methods of contacting cells with external entities both in vivo and ex vivo are well known in the biological arts. For example, contacting a nanoparticle composition and a cell disposed within a mammal can be performed by varied routes of administration (e.g., intravenous, intramuscular, intradermal, and subcutaneous) and can involve varied amounts of nanoparticle compositions. Moreover, more than one cell can be contacted by a nanoparticle composition.

A further example of contacting is between a nanoparticle and a lipid amine. Contacting a nanoparticle (e.g., filled with payload or empty) and a lipid amine can mean that the surface of the nanoparticle is put in physical connection with the lipid amine so that, the lipid amine can form an interaction with the nanoparticle. In some embodiments, contacting a nanoparticle and a lipid amine results in intercalation of the lipid amine into the nanoparticle, for example, starting at the surface of the nanoparticle. In some embodiments, the terms “layering,” “coating,” and “post addition” and “addition” can be used to mean “contacting” in reference to contacting a nanoparticle with a lipid amine.

As used herein, the term “delivering” means providing an entity to a destination. For example, delivering a polynucleotide to a subject can involve administering a nanoparticle composition including the polynucleotide to the subject (e.g., by an intravenous, intramuscular, intradermal, or subcutaneous route). Administration of a nanoparticle composition to a mammal or mammalian cell can involve contacting one or more cells with the nanoparticle composition.

As used herein, “delivery agent” refers to any substance that facilitates, at least in part, the in vivo, in vitro, or ex vivo delivery of a polynucleotide to targeted cells.

As used herein, the term “diastereomer,” means stereoisomers that are not mirror images of one another and are non-superimposable on one another.

As used herein, the term “effective amount” of an agent is that amount sufficient to effect beneficial or desired results, for example, clinical results, and, as such, an “effective amount” depends upon the context in which it is being applied. For example, in the context of administering an agent that treats a protein deficiency, an effective amount of an agent is, for example, an amount of mRNA expressing sufficient protein to ameliorate, reduce, eliminate, or prevent the signs and symptoms associated with the protein deficiency, as compared to the severity of the symptom observed without administration of the agent. The term “effective amount” can be used interchangeably with “effective dose,” “therapeutically effective amount,” or “therapeutically effective dose.”

As used herein, the term “enantiomer” means each individual optically active form of a compound of the present disclosure, having an optical purity or enantiomeric excess (as determined by methods standard in the art) of at least 80% (i.e., at least 90% of one enantiomer and at most 10% of the other enantiomer), at least 90%, or at least 98%.

As used herein, the term “encapsulate” means to enclose, surround, incorporate, or encase.

As used herein, “encapsulation efficiency” refers to the amount of a polynucleotide that becomes part of a nanoparticle composition, relative to the initial total amount of polynucleotide used in the preparation of a nanoparticle composition. For example, if 97 mg of polynucleotide are encapsulated in a nanoparticle composition out of a total 100 mg of polynucleotide initially provided to the composition, the encapsulation efficiency can be given as 97%. As used herein, “encapsulation” can refer to complete, substantial, or partial enclosure, confinement, surrounding, or encasement.

As used herein, “epithelial cells” include cells derived from epithelium. Example epithelial cells are respiratory epithelial cells, nasal epithelial cells, alveolar epithelial cells, lung epithelial cells, or bronchial epithelial cells. In some embodiments, the epithelial cells are human bronchial epithelial (HBE) cells. In some embodiments, epithelial cells are in vitro cells. In some embodiments, epithelial cells are in vivo cells.

As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

As used herein, the term “ex vivo” refers to events that occur outside of an organism (e.g., animal, plant, or microbe or cell or tissue thereof). Ex vivo events can take place in an environment minimally altered from a natural (e.g., in vivo) environment.

As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically the helper lipid is a phospholipid. A function of the helper lipid is to “complement” the amino lipid and increase the fusogenicity of the bilayer and/or to help facilitate endosomal escape, e.g., of nucleic acid delivered to cells. Helper lipids are also believed to be a key structural component to the surface of the LNP.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

The term “ionizable amino lipid” includes those lipids described herein throughout that, for example, exhibit one, two, three, or more fatty acid or fatty alkyl chains and at least one pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to DLin-MC3-DMA (MC3) and (13Z,165Z)—N,N-dimethyl-3-nonydocosa-13-16-dien-1-amine (L608).

As used herein, the term “isomer” means any tautomer, stereoisomer, enantiomer, or diastereomer of any compound of the present disclosure. It is recognized that the compounds of the present disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the present disclosure, the chemical structures depicted herein, and therefore the compounds of the present disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the present disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, a “lipid nanoparticle core” is a lipid nanoparticle to which post addition layers of additional components can be added, such as a lipid amine and/or a PEG-lipid or other lipid. In some embodiments, the lipid nanoparticle core comprises: (i) an ionizable lipid, (ii) a phospholipid, (iii) a structural lipid, and (iv) optionally a PEG-lipid. In further embodiments, the lipid nanoparticle core comprises: (i) an ionizable lipid, (ii) a phospholipid, (iii) a structural lipid, and (iv) a PEG-lipid. In some embodiments, the lipid nanoparticle core can contain payload.

As used herein, a “linker” or “linker structure” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl) phosphine (TCEP), or other reducing agents, and/or photolysis, as well as an ester bond can be cleaved for example by acidic or basic hydrolysis.

As used herein, “lung cells” include cells derived from the lungs. Lung cells can be, for example, lung epithelial cells, airway basal cells, bronchiolar exocrine cells, pulmonary neuroendocrine cells, alveolar cells, or airway epithelial cells. In some embodiments, lung cells are in vitro cells. In some embodiments, lung cells are in vivo cells.

The term “nucleic acid,” in its broadest sense, includes any compound and/or substance that comprises a polymer of nucleotides. These polymers are often referred to as polynucleotides. Exemplary nucleic acids or polynucleotides of the present disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

As used herein, “patient” refers to a subject who can seek or be in need of treatment, requires treatment, is receiving treatment, will receive treatment, or a subject who is under care by a trained professional for a particular disease or condition.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration.

The “pharmaceutically acceptable salts” of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “solvate,” as used herein, means a compound of the present disclosure wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. When water is the solvent, the solvate is referred to as a “hydrate.”

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

As used herein, the term “preventing” refers to partially or completely delaying onset of an infection, disease, disorder and/or condition; partially or completely delaying onset of one or more signs and symptoms, features, or clinical manifestations of a particular infection, disease, disorder, and/or condition; partially or completely delaying onset of one or more signs and symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, “prophylactic” refers to a therapeutic or course of action used to prevent the spread of disease.

The term “salt” includes any anionic and cationic complex. Pharmaceutically acceptable salts represent a subset of non-toxic salts as described hereinabove.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

An individual who is “suffering from” a disease, disorder, and/or condition has been diagnosed with or displays one or more signs and symptoms of the disease, disorder, and/or condition.

An individual who is “susceptible to” a disease, disorder, and/or condition has not been diagnosed with and/or cannot exhibit signs and symptoms of the disease, disorder, and/or condition but harbors a propensity to develop a disease or its signs and symptoms. In some embodiments, an individual who is susceptible to a disease, disorder, and/or condition (for example, cancer) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “synthetic” means produced, prepared, and/or manufactured by the hand of man. Synthesis of polynucleotides or other molecules of the present disclosure can be chemical or enzymatic.

The term “therapeutic or prophylactic agent” refers to an agent that, when administered to a subject, has a therapeutic, diagnostic, and/or prophylactic effect and/or elicits a desired biological and/or pharmacological effect. For example, an mRNA encoding a polypeptide can be a therapeutic or prophylactic agent.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve signs and symptoms of, diagnose, prevent, and/or delay the onset of the infection, disease, disorder, and/or condition.

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more signs and symptoms or features of a disease. For example, “treating” a disease can refer to diminishing signs and symptoms associated with the disease, prolonging the lifespan (increase the survival rate) of patients, reducing the severity of the disease, preventing or delaying the onset of the disease, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. As used herein, the term “Cₙ₋ₘ hydroxyalkyl” refers to an alkyl group substituted with a hydroxy (—OH) group.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphthyl.

As used herein, “halo” refers to F, Cl, Br, or I. In some embodiments, a halo is F, Cl, or Br.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, a “bridged ring” or a “bridged ring group” is cyclic system with at least two joined rings that share three or more atoms. A bridged ring can be a carbocycle ring or a heterocycloalkyl ring. Example bridged rings include

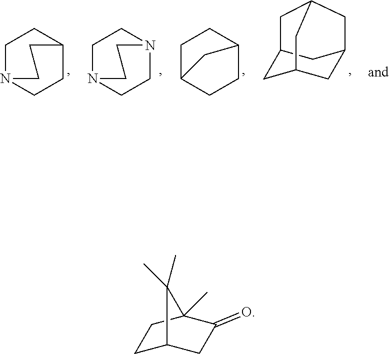

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the present disclosure (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Abbreviations

ACN: acetonitrile
DCM: dichloromethane
EtOAc: ethyl acetate
Hrs: hours
h: hour/hours
LCMS: Liquid chromatography-mass spectrometry
TLC: thin layer chromatography

### Example 1

### Synthesis of Compounds According to Formula A1

### A. Compound SA44: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate

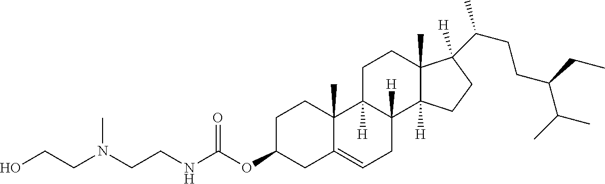

To a solution of β-sitosterol 4-nitrophenyl carbonate (0.120 g, 0.207 mmol) and triethylamine (0.04 mL, 0.3 mmol) in DCM (2.0 mL) was added a solution of 2-[(2-aminoethyl)(methyl)amino]ethanol (0.029 g, 0.25 mmol) in DCM (0.5 mL). The reaction mixture stirred at 40° C. and was monitored by LCMS. At 3 h, the reaction mixture was diluted with DCM and washed with water. The organics were passed through a hydrophobic frit, dried over Na₂SO₄, and concentrated. The crude material was purified via silica gel chromatography (0-12% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate (0.072 g, 0.12 mmol, 58.5%) as a white foam. UPLC/ELSD: RT=2.30 min. MS (ES): m/z=559.6 [M+H]⁺ for C₃₅H₆₂N₂O₃; ¹H NMR (300 MHz, CDCl₃): δ 5.34-5.41 (m, 1H), 4.89 (br. s, 1H), 4.42-4.57 (m, 1H), 3.61 (t, 2H, J=5.4 Hz), 3.28 (dt, 2H, J=5.9, 5.6 Hz), 2.51-2.61 (m, 4H), 2.21-2.41 (m, 2H), 2.28 (s, 3H), 1.76-2.07 (br. m, 5H), 0.88-1.74 (br. m, 22H), 1.01 (s, 3H), 0.92 (d, 3H, J=6.5 Hz), 0.79-0.87 (m, 9H), 0.68 (s, 3H).

### B. Compound SA45: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-(quinuclidin-3-yl)acetate

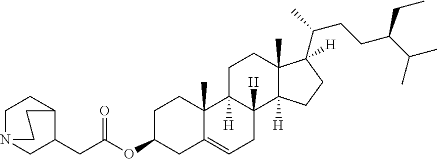

To a stirred solution of β-sitosterol (150 mg, 0.362 mmol), 3-(carboxymethyl)-1-azabicyclo[2.2.2]octan-1-ium chloride (AstaTech, Inc., Bristol, PA) (97 mg, 0.47 mmol), triethylamine (0.08 mL, 0.5 mmol), and 4-(dimethylamino)pyridine (0.022 g, 0.18 mmol) in DCM (2.4 mL) was added 1-ethyl-3-(3-dimethylaminopropyl) carbodiimide hydrochloride (0.104 g, 0.543 mmol). The reaction mixture stirred at rt and was monitored by LCMS. At 15 h, water (ca. 2.5 mL) was added, and the biphasic mixture stirred for 5 min. After this time, the layers were separated, and the aqueous was extracted with DCM (2×) and 9:1 DCM/MeOH. The combined organics were dried over Na₂SO₄ and concentrated. The crude material was purified via silica gel chromatography (0-20% (10% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-(quinuclidin-3-yl)acetate (0.106 g, 0.170 mmol, 47.1%) as a white solid. UPLC/ELSD: RT=2.57 min. MS (ES): m/z=566.6 [M+H]⁺ for C₃₈H₆₃NO₂; ¹H NMR (300 MHz, CDCl₃): δ 5.34-5.41 (m, 1H), 4.55-4.69 (m, 1H), 3.08-3.21 (m, 1H), 2.72-2.93 (m, 4H), 2.25-2.44 (br. m, 5H), 1.76-2.17 (br. m, 6H), 0.89-1.73 (br. m, 27H), 1.02 (s, 3H), 0.92 (d, 3H, J=6.5 Hz), 0.78-0.88 (m, 9H), 0.68 (s, 3H).

### C. Compound SA46: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-(dimethylamino)propyl)carbamate

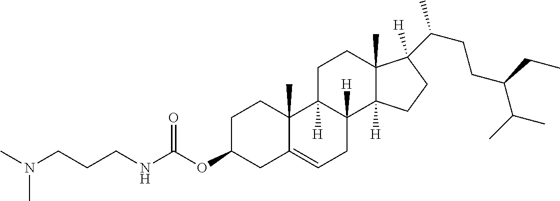

β-Sitosterol 4-nitrophenyl carbonate (0.150 g, 0.259 mmol), triethylamine (0.06 mL, 0.4 mmol), and dimethylaminopropylamine (0.04 mL, 0.3 mmol) were combined in CHCl₃ (2.4 mL) and stirred at 50° C. The reaction was monitored by TLC. At 21.5 hrs, the reaction mixture was cooled to rt and diluted with DCM (10 mL). The organics were washed with 5% aq. NaHCO₃ solution. The aqueous layer was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-(dimethylamino)propyl)carbamate (0.124 g, 0.218 mmol, 84.1%) as a white foam. UPLC/ELSD: RT=2.51 min. MS (ES): m/z=543.1 [M+H]⁺ for C₃₅H₆₂N₂O₂; ¹H NMR (300 MHz, CDCl₃): δ 5.28-5.40 (m, 2H), 4.42-4.56 (m, 1H), 3.23 (dt, 2H, J=6.4, 6.0 Hz), 2.17-2.42 (m, 4H), 2.22 (s, 6H), 1.77-2.05 (br. m, 5H), 0.88-1.72 (br. m, 24H), 1.01 (s, 3H), 0.92 (d, 3H, J=6.5 Hz), 0.77-0.88 (m, 9H), 0.68 (s, 3H).

### D. Compound SA47: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(dimethylamino)butyl)carbamate

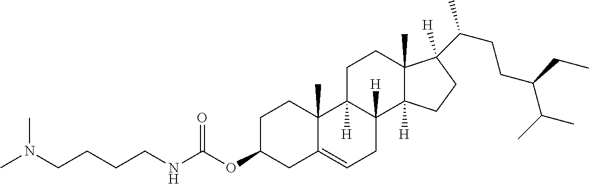

β-Sitosterol 4-nitrophenyl carbonate (0.150 g, 0.259 mmol), triethylamine (0.06 mL, 0.4 mmol), and (4-aminobutyl)dimethylamine (0.05 mL, 0.4 mmol) were combined in CHCl₃ (2.4 mL) and stirred at 50° C. The reaction was monitored by TLC. At 21.5 hrs, the reaction mixture was cooled to rt and diluted with DCM (10 mL). The organics were washed with 5% aq. NaHCO₃ solution. The aqueous layer was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(dimethylamino)butyl)carbamate (0.138 g, 0.232 mmol, 89.7%) as a white foam. UPLC/ELSD: RT=2.56 min. MS (ES): m/z=557.3 [M+H]⁺ for C₃₆H₆₄N₂O₂; ¹H NMR (300 MHz, CDCl₃): δ 5.34-5.40 (m, 1H), 5.09 (m, 1H), 4.41-4.56 (m, 1H), 3.17 (dt, 2H, J=5.9, 5.8 Hz), 2.17-2.44 (m, 4H), 2.21 (s, 6H), 1.77-2.05 (br. m, 5H), 0.88-1.73 (br. m, 26H), 1.01 (s, 3H), 0.92 (d, 3H, J=6.5 Hz), 0.77-0.87 (m, 9H), 0.68 (s, 3H).

### E. Compound SA48: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(bis(2-hydroxyethyl)amino)ethyl)carbamate

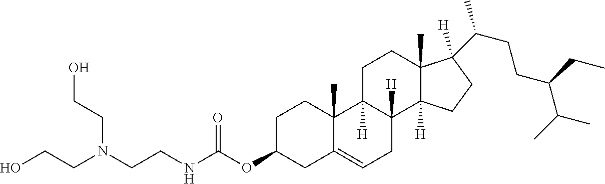

β-Sitosterol 4-nitrophenyl carbonate (0.150 g, 0.259 mmol), triethylamine (0.06 mL, 0.4 mmol), and 2-[(2-aminoethyl) (2-hydroxyethyl)amino]ethanol (0.05 mL, 0.4 mmol) were combined in CHCl₃ (2.5 mL) and stirred at 50° C. The reaction was monitored by TLC. At 21.5 hrs, the reaction mixture was cooled to rt and diluted with DCM (10 mL). The organics were washed with a 5% aq. NaHCO₃ solution. The aqueous layer was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(bis(2-hydroxyethyl)amino)ethyl)carbamate (0.056 g, 0.087 mmol, 33.6%) as a white solid. UPLC/ELSD: RT=2.43 min. MS (ES): m/z=589.2 [M+H]⁺ for C₃₆H₆₄N₂O₄; ¹H NMR (300 MHz, CDCl₃): δ 5.33-5.41 (m, 1H), 5.21 (br. s, 1H), 4.42-4.58 (m, 1H), 3.62 (t, 4H, J=5.0 Hz), 3.01-3.38 (br. m, 4H), 2.59-2.76 (m, 6H), 2.20-2.42 (m, 2H), 1.76-2.06 (br. m, 5H), 0.88-1.72 (br. m, 22H), 1.00 (s, 3H), 0.92 (d, 3H, J=6.4 Hz), 0.78-0.87 (m, 9H), 0.67 (s, 3H).

### F. Compound SA49: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(2-(dimethylamino)ethoxy)ethyl)carbamate

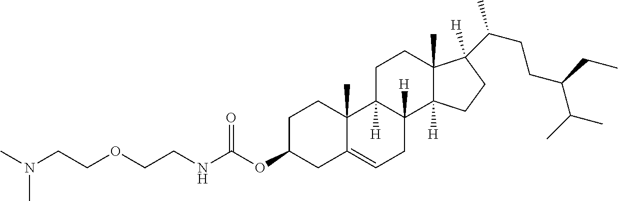

To a solution of β-sitosterol 4-nitrophenyl carbonate (0.150 g, 0.259 mmol), triethylamine (0.06 mL, 0.4 mmol) in CHCl₃ (2.5 mL) was added a solution of [2-(2-aminoethoxy)ethyl]dimethylamine (0.049 g, 0.37 mmol) in CHCl₃ (0.5 mL). The reaction mixture stirred at 50° C. and was monitored by TLC. At 21.5 hrs, the reaction mixture was cooled to rt and diluted with DCM (10 mL). The organics were washed with a 5% aq. NaHCO₃ solution. The aqueous was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(2-(dimethylamino)ethoxy)ethyl)carbamate (0.123 g, 0.201 mmol, 77.8%) as a white foam. UPLC/ELSD: RT=2.55 min. MS (ES): m/z=573.3 [M+H]⁺ for C₃₆H₆₄N₂O₃; ¹H NMR (300 MHz, CDCl₃): δ 5.46-5.57 (m, 1H), 5.33-5.41 (m, 1H), 4.41-4.58 (m, 1H), 3.55 (t, 2H, J=5.6 Hz), 3.53 (t, 2H, J=4.5 Hz), 3.35 (dt, 2H, J=5.1, 5.0 Hz), 2.49 (t, 2H, J=5.6 Hz), 2.21-2.42 (m, 2H), 2.27 (s, 6H), 1.76-2.07 (br. m, 5H), 0.88-1.74 (br. m, 22H), 1.01 (s, 3H), 0.92 (d, 3H, J=6.4 Hz), 0.78-0.87 (m, 9H), 0.68 (s, 3H).

### G. Compound SA52: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-(4,7-dimethyl-1,4,7-triazonan-1-yl)-4-oxobutanoate

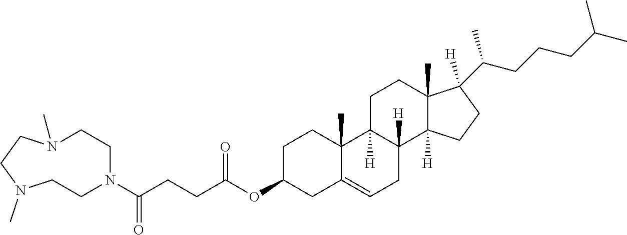

To a stirred solution of cholesteryl hemisuccinate (0.120 g, 0.247 mmol), 1,4-dimethyl-1,4,7-triazonane (Enamine, Monmouth Junction, NJ) (0.042 g, 0.27 mmol), and DMAP (cat.) in DCM (1.5 mL) was added 1-ethyl-3-(3-dimethylaminopropyl) carbodiimide hydrochloride (0.071 g, 0.37 mmol). The reaction mixture stirred at rt and was monitored by LCMS. At 46 h, water (2 mL) was added. The mixture stirred at rt for 16 h, then was diluted with 5% aq. NaHCO₃ solution (5 mL) and then extracted with DCM (3×10 mL). The combined organics were passed through a hydrophobic frit, dried over Na₂SO₄, and concentrated. The crude material was purified via silica gel chromatography (0-12% (5% conc. aq. NH₄OH in MeOH) in DCM). The material was purified again via silica gel chromatography (0-10% (5% conc. aq. NH₄OH in MeOH) in DCM) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(4,7-dimethyl-1,4,7-triazonan-1-yl)-4-oxobutanoate (0.100 g, 0.140 mmol, 56.7%) as a clear oil. UPLC/ELSD: RT=2.52 min. MS (ES): m/z=626.2 [M+H]⁺ for C₃₉H₆₇N₃O₃; ¹H NMR (300 MHz, CDCl₃): δ 5.31-5.40 (m, 1H), 4.52-4.69 (m, 1H), 3.40-3.58 (m, 4H), 2.89-2.98 (m, 2H), 2.71-2.81 (m, 2H), 2.61-2.68 (m, 4H), 2.45-2.53 (m, 4H), 2.27-2.42 (m, 2H), 2.40 (s, 3H), 2.35 (s, 3H), 1.74-2.04 (br. m, 5H), 0.93-1.68 (m, 21H), 1.00 (s, 3H), 0.90 (d, 3H, J=6.4 Hz), 0.86 (d, 3H, J=6.6 Hz), 0.85 (d, 3H, J=6.6 Hz), 0.67 (s, 3H).

### H. Compound SA53: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)-2-methylpropyl)carbamate

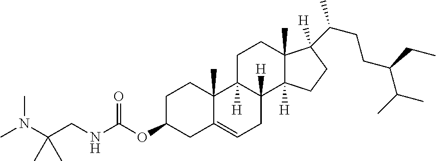

β-Sitosterol 4-nitrophenyl carbonate (0.150 g, 0.259 mmol), (1-amino-2-methylpropan-2-yl)dimethylamine (0.036 g, 0.31 mmol), and triethylamine (0.06 mL, 0.4 mmol) were combined in CHCl₃ (2.4 mL) and stirred at 50° C. The reaction was monitored by TLC. At 28 h, triethylamine (0.03 mL) and (1-amino-2-methylpropan-2-yl)dimethylamine (22 mg) were added. The reaction mixture stirred at 55° C. At 46 h, the reaction mixture was cooled to rt, diluted with a 5% aq. NaHCO₃ solution (10 mL), and extracted with DCM (2×10 mL). The combined organics were passed through a hydrophobic frit, dried over Na₂SO₄, and concentrated. The crude material was purified via silica gel chromatography (0-15% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)-2-methylpropyl)carbamate (0.091 g, 0.16 mmol, 60.4%) as an off-white solid. UPLC/ELSD: RT=2.55 min. MS (ES): m/z=557.4 [M+H]⁺ for C₃₆H₆₄N₂O₂; ¹H NMR (300 MHz, CDCl₃): δ 5.34-5.43 (m, 1H), 5.12-5.31 (m, 1H), 4.42-4.59 (m, 1H), 3.09 (m, 2H), 2.20-2.43 (m, 2H), 2.17 (s, 6H), 1.75-2.06 (br. m, 5H), 0.88-1.74 (br. m, 22H), 1.01 (s, 3H), 0.99 (s, 6H), 0.92 (d, 3H, J=6.5 Hz), 0.77-0.88 (m, 9H), 0.68 (s, 3H).

### I. Compound SA54: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl ((1-(dimethylamino)cyclopropyl)methyl)carbamate

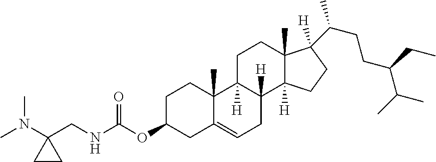

β-Sitosterol 4-nitrophenyl carbonate (0.150 g, 0.259 mmol), 1-(aminomethyl)-N,N-dimethylcyclopropan-1-amine (0.035 g, 0.31 mmol) and triethylamine (0.06 mL, 0.4 mmol) were combined in CHCl₃ (2.4 mL) and stirred at 50° C. The reaction was monitored by TLC. At 28 hrs, triethylamine (0.03 mL) and 1-(aminomethyl)-N,N-dimethylcyclopropan-1-amine (22 mg) were added. The reaction mixture stirred at 55° C. At 46 hrs, the reaction mixture was cooled to rt, diluted with 5% aq. NaHCO₃ solution (10 mL), and then extracted with DCM (2×10 mL). The combined organics were passed through a hydrophobic frit, dried over Na₂SO₄, and concentrated. The crude material was purified via silica gel chromatography (0-15% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl ((1-(dimethylamino)cyclopropyl)methyl)carbamate (0.130 g, 0.225 mmol, 86.9%) as an off-white solid. UPLC/ELSD: RT=2.57 min. MS (ES): m/z=555.3 [M+H]⁺ for C₃₆H₆₂N₂O₂; ¹H NMR (300 MHz, CDCl₃): δ 5.34-5.41 (m, 1H), 4.68 (br. s, 1H), 4.41-4.57 (m, 1H), 3.28 (d, 2H, J=4.9 Hz), 2.16-2.57 (m, 2H), 2.40 (s, 6H), 1.76-2.08 (br. m, 5H), 0.88-1.74 (br. m, 22H), 1.01 (s, 3H), 0.92 (d, 3H, J=6.5 Hz), 0.77-0.87 (m, 9H), 0.68 (s, 3H), 0.65 (br. s, 2H), 0.53 (br. s, 2H).

### J. Compound SA55: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-amino-2-methylpropyl)carbamate Hydrochloride

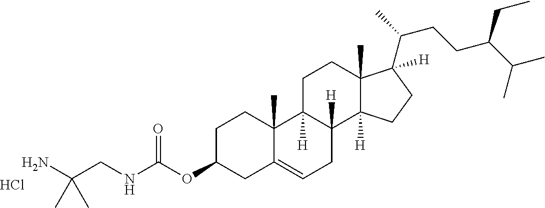

### Step 1: tert-Butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) (2,2-dimethylethane-1,2-diyl)dicarbamate

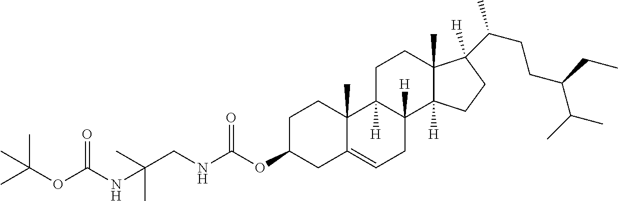

β-Sitosterol 4-nitrophenyl carbonate (0.225 g, 0.388 mmol), tert-butyl N-(1-amino-2-methylpropan-2-yl)carbamate (0.088 g, 0.47 mmol) and triethylamine (0.08 mL, 0.6 mmol) were combined in CHCl₃ (3.6 mL) and stirred at 50° C. The reaction was monitored by TLC. At 28 h, triethylamine (0.04 mL) and tert-butyl N-(1-amino-2-methylpropan-2-yl)carbamate (41 mg) were added. The reaction mixture stirred at 55° C. At 46 hrs, the reaction mixture was cooled to rt, diluted with 5% aq. NaHCO₃ solution (10 mL), and then extracted with DCM (2×10 mL). The combined organics were passed through a hydrophobic frit, dried over Na₂SO₄, and concentrated. The crude material was purified via silica gel chromatography (0-20% (5% conc. aq. NH₄OH in MeOH) in DCM) to afford tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) (2,2-dimethylethane-1,2-diyl)dicarbamate (0.220 g, 0.350 mmol, 90.1%) as a white solid. ¹H NMR (300 MHz, CDCl₃): δ 5.34-5.43 (m, 1H), 5.16 (br. s, 1H), 4.41-4.69 (m, 2H), 3.35 (d, 2H, J=6.3 Hz), 2.21-2.43 (m, 2H), 1.76-2.08 (br. m, 5H), 0.88-1.73 (br. m, 22H), 1.43 (s, 9H), 1.25 (s, 6H), 1.01 (s, 3H), 0.92 (d, 3H, J=6.4 Hz), 0.78-0.88 (m, 9H), 0.68 (s, 3H). UPLC/ELSD: RT=3.40 min. MS (ES): m/z=651.1 [M+Na]⁺ for C₃₉H₆₈N₂O₄.

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-amino-2-methylpropyl)carbamate Hydrochloride

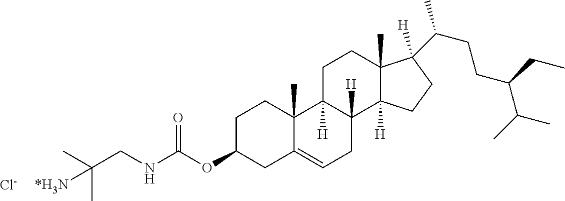

To a solution of tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) (2,2-dimethylethane-1,2-diyl)dicarbamate (0.211 g, 0.335 mmol) in iPrOH (2.1 mL) was added 5-6 N HCl in iPrOH (0.35 mL). The reaction mixture stirred at 40° C. and was monitored by LCMS. At 17 h, the reaction mixture was cooled to rt. ACN (4 mL) was added, and the suspension was cooled to 0° C. in an ice bath. Solids were collected by vacuum filtration and rinsed with 2:1 ACN: iPrOH to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-amino-2-methylpropyl)carbamate hydrochloride (0.174 g, 0.292 mmol, 87.0%) as a white solid. UPLC/ELSD: RT=2.51 min. MS (ES): m/z=529.3 [M+H]⁺ for C₃₄H₆₀N₂O₂; ¹H NMR (300 MHz, CD₃OD): δ 5.36-5.47 (m, 1H), 4.37-4.53 (m, 1H), 3.24 (s, 2H), 2.28-2.46 (m, 2H), 1.81-2.13 (br. m, 5H), 0.92-1.77 (br. m, 22H), 1.31 (s, 6H), 1.06 (s, 3H), 0.96 (d, 3H, J=6.4 Hz), 0.81-0.91 (m, 9H), 0.74 (s, 3H).

### K. Compound SA85: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)carbamate Hydrochloride

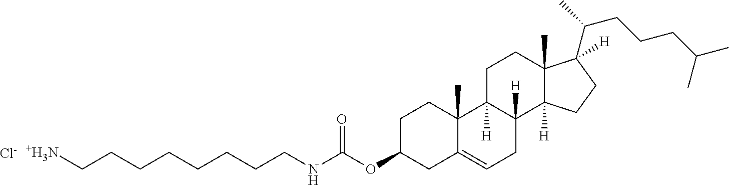

### Step 1: Tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) octane-1,8-diyldicarbamate

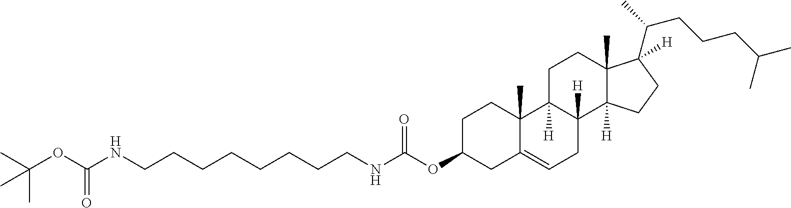

Cholesterol 4-nitrophenyl carbonate (0.200 g, 0.362 mmol), tert-butyl N-(8-aminooctyl)carbamate (0.106 g, 0.435 mmol), triethylamine (0.15 mL, 1.1 mmol) were combined in toluene (3.5 mL). The reaction mixture stirred at 90° C. and was monitored by LCMS. At 18 h, the reaction mixture was cooled to rt, diluted with dichloromethane (25 mL), then washed with 5% aq. NaHCO₃ solution (3×25 mL). The organics were passed through a hydrophobic frit, dried over Na₂SO₄, and concentrated. The crude material was purified via silica gel chromatography (0-30% ethyl acetate in hexanes) to afford tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) octane-1,8-diyldicarbamate (0.236 g, 0.359 mmol, 99.1%) as a clear oil. UPLC/ELSD: RT=3.34 min. MS (ES): m/z=658.36 [M+H]⁺ for C₄₁H₇₂N₂O₄; ¹H NMR (300 MHz, CDCl₃): δ 5.33-5.42 (m, 1H), 4.38-4.66 (m, 3H), 3.03-3.24 (m, 4H), 2.19-2.43 (m, 2H), 1.75-2.17 (m, 5H), 0.94-1.67 (br. m, 33H), 1.44 (s, 9H), 1.01 (s, 3H), 0.91 (d, 3H, J=6.4 Hz), 0.87 (d, 3H, J=6.6 Hz), 0.86 (d, 3H, J=6.5 Hz), 0.68 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)carbamate Hydrochloride

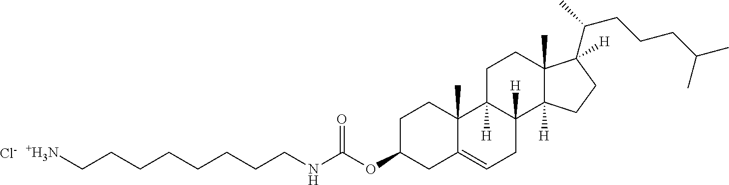

To a solution of tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) octane-1,8-diyldicarbamate (0.236 g, 0.359 mmol) in isopropanol (3.5 mL) was added 5-6 N HCl in isopropanol (0.25 mL). The reaction mixture stirred at 40° C. and was monitored by LCMS. At 16 h, 5-6 N HCl in isopropanol (0.25 mL) was added. At 20 h, acetonitrile (10.5 mL) was added, and the suspension was stirred at rt for 5 min. Then solids were collected by vacuum filtration rinsing with 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)carbamate hydrochloride (0.126 g, 0.208 mmol, 57.9%) as a white solid. UPLC/ELSD: RT=2.62 min. MS (ES): m/z=558.16 [M+H]⁺ for C₃₆H₆₄N₂O₂; ¹H NMR (300 MHz, CD₃OD): δ 5.34-5.44 (m, 1H), 4.28-4.46 (m, 1H), 3.07 (t, 2H, J=6.4 Hz), 2.91 (t, 2H, J=7.0 Hz), 2.22-2.40 (m, 2H), 1.79-2.12 (m, 5H), 0.80-1.74 (br. m, 45H), 0.72 (s, 3H).

### L. Compound SA86: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)carbamate Hydrochloride

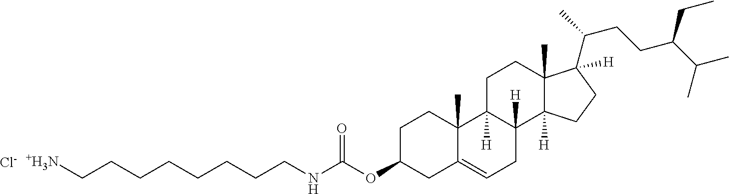

### Step 1: Tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) octane-1,8-diyldicarbamate

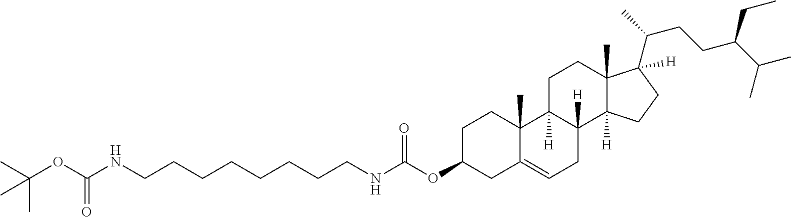

β-Sitosterol 4-nitrophenyl carbonate (0.200 g, 0.345 mmol), tert-butyl N-(8-aminooctyl)carbamate (0.105 g, 0.431 mmol), and triethylamine (0.15 mL, 1.1 mmol) were combined in toluene (3.5 mL). The reaction mixture stirred at 90° C. and was monitored by LCMS. At 18 h, the reaction mixture was cooled to rt, diluted with dichloromethane (25 mL) and then washed with 5% aq. NaHCO₃ solution (3×25 mL). The combined organics were passed through a hydrophobic frit, dried over Na₂SO₄, and concentrated. The crude material was purified via silica gel chromatography (0-30% ethyl acetate in hexanes) to afford tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) octane-1,8-diyldicarbamate (0.199 g, 0.29 mmol, 84.2%) as a white foam. UPLC/ELSD: RT=3.44 min. MS (ES): m/z=629.86 [(M+H)−(CH₃)₂C═CH₂]⁺ for C₄₃H₇₆N₂O₄; ¹H NMR (300 MHz, CDCl₃): δ 5.33-5.45 (m, 1H), 4.30-4.71 (m, 3H), 2.99-3.28 (m, 4H), 2.18-2.45 (m, 2H), 1.76-2.11 (m, 5H), 0.88-1.73 (br. m, 34H), 1.44 (s, 9H), 1.01 (s, 3H), 0.92 (d, 3H, J=6.4 Hz), 0.77-0.88 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)carbamate Hydrochloride

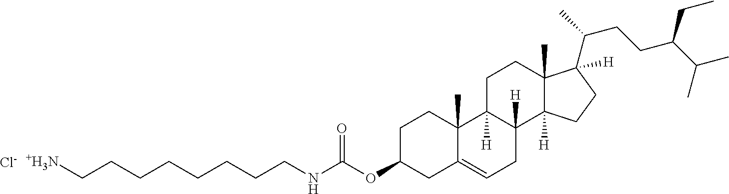

To a solution of tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) octane-1,8-diyldicarbamate (0.188 g, 0.274 mmol) in isopropanol (2.8 mL) was added 5-6 N HCl in isopropanol (0.20 mL). The reaction mixture stirred at 40° C. and was monitored by LCMS. At 16 h, 5-6 N HCl in isopropanol (0.20 mL) was added. At 20 h, acetonitrile (8.4 mL) was added, and the suspension was stirred at rt for 5 min. Then solids were collected by vacuum filtration rinsing with 3:1 acetonitrile/isopropanol to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)carbamate hydrochloride (0.107 g, 0.162 mmol, 59.2%) as a white solid. UPLC/ELSD: RT=2.73 min. MS (ES): m/z=585.68 [M+H]⁺ for C₃₈H₆₈N₂O₂; ¹H NMR (300 MHz, CD₃OD): δ 5.33-5.45 (m, 1H), 4.27-4.47 (m, 1H), 3.07 (t, 2H, J=6.3 Hz), 2.91 (t, 2H, J=6.6 Hz), 2.21-2.40 (m, 2H), 1.79-2.12 (m, 5H), 0.79-1.77 (br. m, 49H), 0.73 (s, 3H).

### M. Compound SA91: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((2-aminoethyl)amino)-2-oxoethyl)disulfaneyl)acetate Hydrochloride

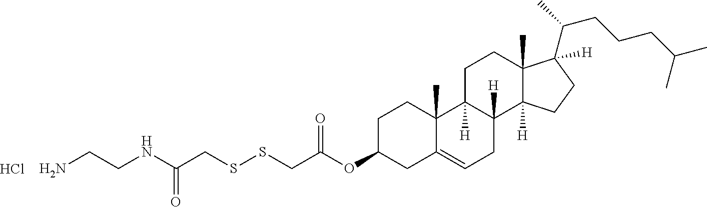

### Step 1: 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic Acid

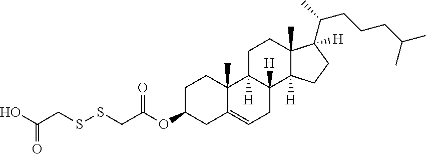

To a solution of cholesterol (5.00 g, 12.93 mmol) in dry DCM (100 mL) stirring under nitrogen was added dithiodiglycolic acid (4.53 mL, 25.86 mmol). The solution was then cooled to 0° C., and dimethylaminopyridine (0.32 g, 2.59 mmol) and 1-ethyl-3-(3-dimethylaminopropyl) carbodiimide hydrochloride (4.96 g, 25.86 mmol) were added, followed by dropwise addition of triethylamine (4.52 mL, 25.86 mmol). The solution was allowed to gradually warm to room temperature and stir overnight. The following day, the solution was washed with saturated sodium bicarbonate (1×25 mL) and water (1×25 mL), dried over sodium sulfate, filtered, and concentrated to a brown oil. The oil was taken up in DCM and purified on silica in hexanes with a 0-100% EtOAc gradient. Product-containing fractions were pooled and concentrated to give 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid as a dark brown solid (3.76 g, 6.82 mmol, 52.7%). UPLC/ELSD: RT: 3.11 min. MS (ES): m/z (MH⁺) 551.8 for C₃₁H₅₀O₄S₂. ¹H NMR (300 MHz, CDCl₃) δ: ppm 9.04 (br. s, 1H), 5.41 (m, 1H), 4.69 (br. m, 1H), 3.65 (s, 2H), 3.60 (s, 1H), 2.39 (d, 2H, J=9 Hz), 2.01 (br. m, 5H), 1.52 (br. m, 11H), 1.16 (br. m, 6H), 1.04 (s, 6H), 0.95 (d, 3H, J=6 Hz), 0.86 (d, 6H, J=6 Hz), 0.70 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 13,13-dimethyl-6,11-dioxo-12-oxa-3,4-dithia-7,10-diazatetradecanoate

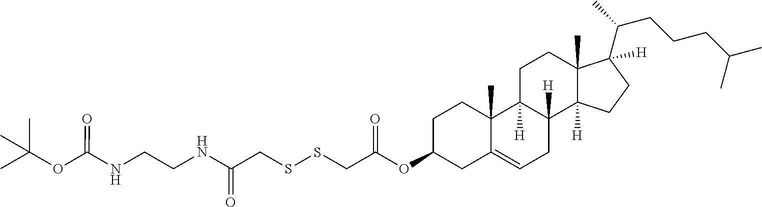

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.31 g, 0.57 mmol) in dry DCM (5 mL) stirring under nitrogen was added tert-butyl (2-aminoethyl)carbamate (0.14 mL, 0.85 mmol), dimethylaminopyridine (0.01 g, 0.06 mmol), 1-ethyl-3-(3-dimethylaminopropyl) carbodiimide hydrochloride (0.16 g, 0.85 mmol), and diisopropylethylamine (0.30 mL, 1.71 mmol). The solution was allowed to stir overnight at room temperature. The following day, the solution was washed with saturated sodium bicarbonate (1×5 mL) and brine (1×5 mL), dried over sodium sulfate, filtered, and concentrated to a white solid. The solid was taken up in DCM and purified on silica in DCM with a 0-60% (75:20:5 DCM/MeOH/aqueous NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 13,13-dimethyl-6,11-dioxo-12-oxa-3,4-dithia-7,10-diazatetradecanoate as a yellow oil (0.06 g, 0.08 mmol, 14.0%). UPLC/ELSD: RT: 3.19 min. MS (ES): m/z (MH⁺) 694.1 for C₃₈H₆₄N₂O₅S₂. ¹H NMR (300 MHz, CDCl₃) δ: ppm 5.40 (m, 1H), 4.72 (br. m, 3H), 4.12 (m, 1H), 3.79 (m, 2H), 3.56 (s, 2H), 3.48 (m, 6H), 3.33 (br. m, 3H), 2.38 (d, 2H, J=9 Hz), 1.88 (br. m, 11H), 1.46 (s, 24H), 1.27 (br. m, 12H), 1.04 (s, 6H), 0.94 (d, 4H, J=6 Hz), 0.89 (d, 6H, J=6 Hz), 0.69 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((2-aminoethyl)amino)-2-oxoethyl)disulfaneyl)acetate Hydrochloride

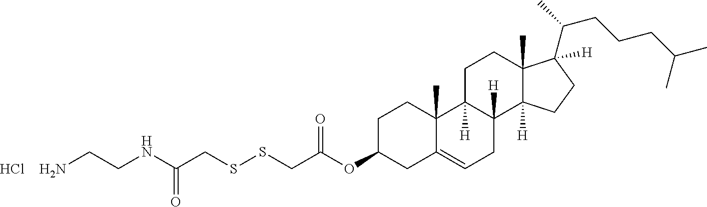

To a solution (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 13,13-dimethyl-6,11-dioxo-12-oxa-3,4-dithia-7,10-diazatetradecanoate (0.09 g, 0.12 mmol) in isopropanol (5 mL) set stirring under nitrogen was added hydrochloric acid (5 N in isopropanol, 0.25 mL, 1.23 mmol) dropwise. The solution was heated to 40° C. and allowed to proceed overnight. The following morning, the solution was cooled to room temperature and dry acetonitrile (10 mL) was added to the mixture. The mixture was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((2-aminoethyl)amino)-2-oxoethyl)disulfaneyl)acetate hydrochloride as a white solid (0.02 g, 0.03 mmol, 26.0%). UPLC/ELSD: RT=2.54 min. MS (ES): m/z (MH⁺) 593.7 for C₃₃H₅₇ClN₂O₃S₂. ¹H NMR (300 MHz, MeOD) δ: ppm 8.41 (br. s, 1H), 5.43 (m, 1H), 4.62 (br. m, 2H), 4.03 (m, 1H), 3.65 (s, 3H), 3.57 (s, 3H), 3.11 (m, 3H), 2.37 (br. m, 2H), 1.93 (br. m, 5H), 1.55 (br. m, 11H), 1.17 (m, 6H), 1.08 (s, 4H), 0.98 (d, 3H, J=6 Hz), 0.91 (d, 5H, J=6 Hz), 0.75 (s, 3H).

### N. Compound SA92: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((6-aminohexyl)amino)-2-oxoethyl)disulfaneyl)acetate Hydrochloride

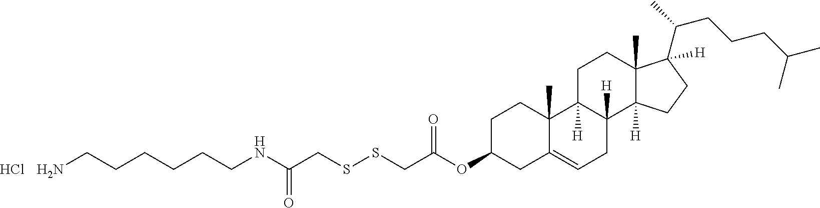

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 17,17-dimethyl-6,15-dioxo-16-oxa-3,4-dithia-7,14-diazaoctadecanoate

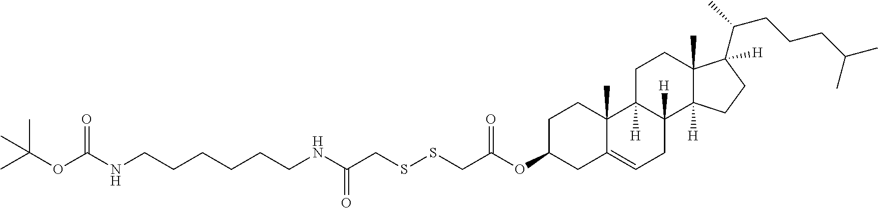

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (6-aminohexyl)carbamate (0.25 mL, 1.09 mmol), dimethylaminopyridine (0.03 g, 0.27 mmol), and 1-ethyl-3-(3-dimethylaminopropyl) carbodiimide hydrochloride (0.21 g, 1.09 mmol). The solution was allowed to stir overnight at room temperature. The following day, the solution was diluted with DCM, washed with saturated sodium bicarbonate (1×10 mL) and brine (1×10 mL), dried over sodium sulfate, filtered, and concentrated to a white solid. The solid was taken up in DCM and purified on silica in hexanes with a 0-80% EtOAc gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 17,17-dimethyl-6,15-dioxo-16-oxa-3,4-dithia-7,14-diazaoctadecanoate as a light yellow oil (0.11 g, 0.14 mmol, 26.2%). UPLC/ELSD: RT: 3.28 min. MS (ES): m/z (MH⁺) 750.1 for C₄₂H₇₂N₂O₅S₂. ¹H NMR (300 MHz, CDCl₃) δ: ppm 6.80 (br. s, 1H), 5.40 (br. m, 1H), 4.66 (br. m, 2H), 3.66 (m, 1H), 3.54 (s, 2H), 3.46 (s, 2H), 3.31 (br. m, 3H), 3.11 (br. m, 3H), 2.37 (d, 2H, J=9 Hz), 2.04 (br. m, 6H), 1.56 (br. m, 7H), 1.44 (s, 21H), 1.35 (br. m, 10H), 1.14 (m, 7H), 1.03 (s, 6H), 0.90 (d, 4H, J=6 Hz), 0.87 (d, 6H, J=6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((6-aminohexyl)amino)-2-oxoethyl)disulfaneyl)acetate Hydrochloride

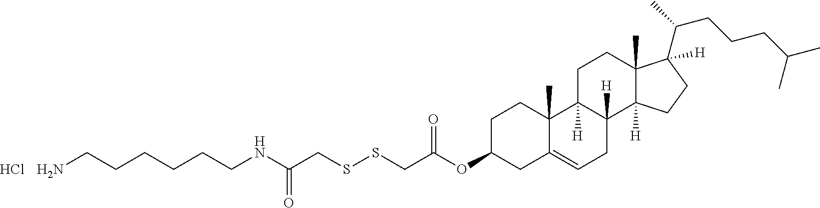

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 17,17-dimethyl-6,15-dioxo-16-oxa-3,4-dithia-7,14-diazaoctadecanoate (0.11 g, 0.14 mmol) in isopropanol (5 mL) set stirring under nitrogen was added hydrochloric acid (5 N in isopropanol, 0.29 mL, 1.43 mmol) dropwise. The solution was heated to 40° C. and allowed to proceed overnight. The following morning, the solution was cooled to room temperature and dry acetonitrile (10 mL) was added to the mixture. The mixture was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((6-aminohexyl)amino)-2-oxoethyl)disulfaneyl)acetate hydrochloride as a white solid (0.04 g, 0.05 mmol, 34.4%). UPLC/ELSD: RT=2.53 min. MS (ES): m/z (MH⁺) 650.0 for C₃₇H₆₅ClN₂O₃S₂. ¹H NMR (300 MHz, MeOD) δ: ppm 5.32 (m, 1H), 4.49 (br. m, 1H), 3.83 (m, 1H), 3.58 (m, 2H), 3.52 (s, 2H), 3.38 (s, 2H), 3.22 (m, 5H), 3.14 (br. m, 3H), 2.83 (t, 5H), 2.25 (m, 2H), 1.86 (br. m, 7H), 1.58 (m, 19H), 1.33 (br. m, 17H), 1.06 (d, 13H, J=6 Hz), 0.96 (s, 7H), 0.86 (d, 5H, J=6 Hz), 0.80 (d, 8H, J=6 Hz), 0.63 (s, 4H).

### O. Compound SA93: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((8-aminooctyl)amino)-2-oxoethyl)disulfaneyl)acetate Hydrochloride

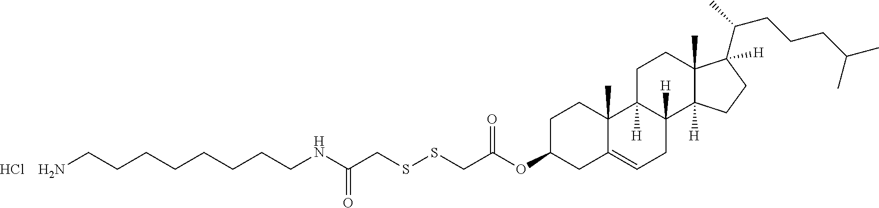

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 19,19-dimethyl-6,17-dioxo-18-oxa-3,4-dithia-7,16-diazaicosanoate

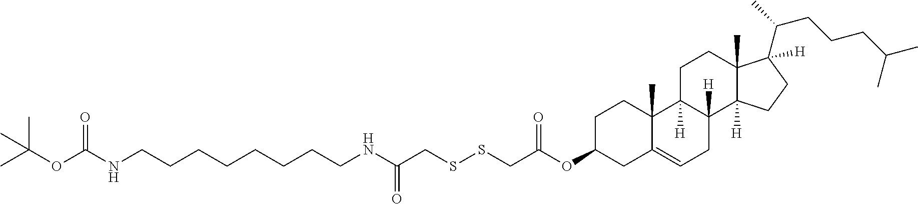

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (8-aminooctyl)carbamate (0.27 mL, 1.09 mmol), dimethylaminopyridine (0.03 g, 0.27 mmol), and 1-ethyl-3-(3-dimethylaminopropyl) carbodiimide hydrochloride (0.21 g, 1.09 mmol). The solution was allowed to stir overnight at room temperature. The following day, the solution was diluted with DCM, washed with saturated sodium bicarbonate (1×10 mL) and brine (1×10 mL), dried over sodium sulfate, filtered, and concentrated to a white solid. The solid was taken up in DCM and purified on silica in hexanes with a 0-80% EtOAc gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 19,19-dimethyl-6,17-dioxo-18-oxa-3,4-dithia-7,16-diazaicosanoate as a light yellow oil (0.11 g, 0.14 mmol, 26.9%). UPLC/ELSD: RT: 3.34 min. MS (ES): m/z (MH⁺) 778.1 for C₄₄H₇₆N₂O₅S₂. ¹H NMR (300 MHz, CDCl₃) δ: ppm 6.75 (br. s, 1H), 5.40 (br. m, 1H), 4.66 (br. m, 2H), 3.54 (s, 2H), 3.46 (s, 2H), 3.28 (br. m, 2H), 3.08 (br. m, 2H), 2.37 (d, 2H, J=9 Hz), 1.91 (br. m, 6H), 1.44 (br. s, 22H), 1.31 (br. m, 13H), 1.11 (m, 7H), 1.03 (s, 6H), 0.93 (d, 4H, J=6 Hz), 0.88 (d, 6H, J=6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((8-aminooctyl)amino)-2-oxoethyl)disulfaneyl)acetate Hydrochloride

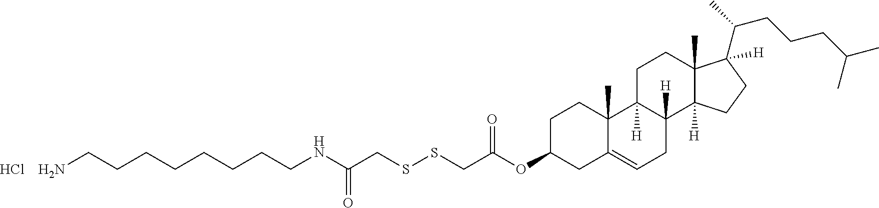

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 19,19-dimethyl-6,17-dioxo-18-oxa-3,4-dithia-7,16-diazaicosanoate (0.11 g, 0.15 mmol) in isopropanol (5 mL) set stirring under nitrogen was added hydrochloric acid (5 N in isopropanol, 0.29 mL, 1.47 mmol) dropwise. The solution was heated to 40° C. and allowed to proceed overnight. The following morning, the solution was cooled to room temperature and dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((8-aminooctyl)amino)-2-oxoethyl)disulfaneyl)acetate hydrochloride as a white solid (0.03 g, 0.04 mmol, 26.7%). UPLC/ELSD: RT=2.52 min. MS (ES): m/z (MH⁺) 677.9 for C₃₉H₆₉ClN₂O₃S₂. ¹H NMR (300 MHz, MeOD) δ: ppm 5.31 (m, 1H), 4.48 (br. m, 1H), 3.82 (m, 1H), 3.51 (s, 2H), 3.36 (s, 2H), 3.21 (m, 7H), 3.12 (br. m, 2H), 2.81 (t, 2H), 2.27 (m, 2H), 1.94 (br. m, 11H), 1.53 (m, 18H), 1.28 (br. m, 15H), 1.04 (d, 14H, J=6 Hz), 0.96 (m, 10H), 0.86 (d, 6H, J=6 Hz), 0.80 (d, 10H, J=6 Hz), 0.63 (s, 5H).

### P. Compound SA94: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((10-aminodecyl)amino)-2-oxoethyl)disulfaneyl)acetate Hydrochloride

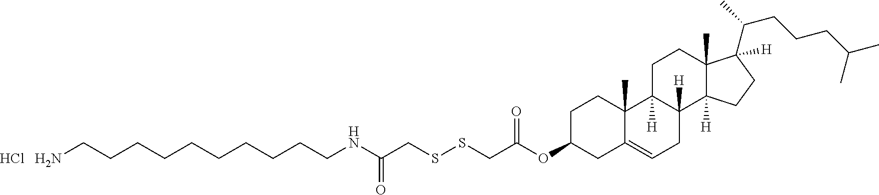

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 21,21-dimethyl-6,19-dioxo-20-oxa-3,4-dithia-7,18-diazadocosanoate

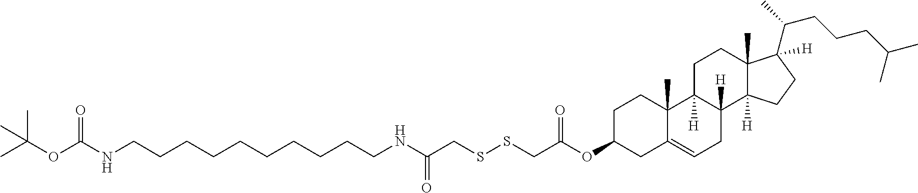

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (10-aminodecyl)carbamate (0.31 g, 1.09 mmol), dimethylaminopyridine (0.03 g, 0.27 mmol), and 1-ethyl-3-(3-dimethylaminopropyl) carbodiimide hydrochloride (0.21 g, 1.09 mmol). The solution was allowed to stir overnight at room temperature. The following day, the solution was diluted with DCM, washed with saturated sodium bicarbonate (1×10 mL) and brine (1×10 mL), dried over sodium sulfate, filtered, and concentrated to a white solid. The solid was taken up in DCM and purified on silica in hexanes with a 0-80% EtOAc gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 21,21-dimethyl-6,19-dioxo-20-oxa-3,4-dithia-7,18-diazadocosanoate as a light yellow oil (0.14 g, 0.17 mmol, 31.5%). UPLC/ELSD: RT: 3.43 min. MS (ES): m/z (MH⁺) 806.3 for C₄₆H₈₀N₂O₅S₂. ¹H NMR (300 MHz, CDCl₃) δ: ppm 6.75 (br. s, 1H), 5.40 (br. m, 1H), 4.67 (br. m, 2H), 3.53 (s, 2H), 3.46 (s, 2H), 3.28 (br. m, 2H), 3.10 (br. m, 2H), 2.34 (d, 2H, J=9 Hz), 2.00 (br. m, 5H), 1.44 (br. s, 20H), 1.27 (br. m, 16H), 1.11 (m, 7H), 1.03 (s, 6H), 0.90 (d, 4H, J=6 Hz), 0.87 (d, 6H, J=6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((10-aminodecyl)amino)-2-oxoethyl)disulfaneyl)acetate Hydrochloride

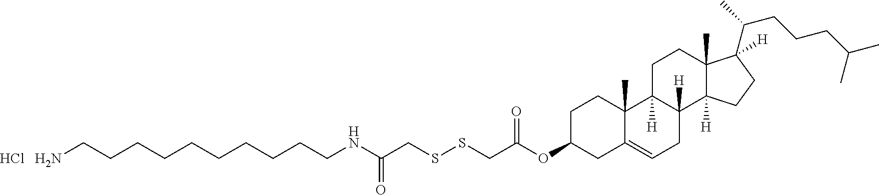

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 21,21-dimethyl-6,19-dioxo-20-oxa-3,4-dithia-7,18-diazadocosanoate (0.14 g, 0.17 mmol) in isopropanol (5 mL) set stirring under nitrogen was added hydrochloric acid (5 N in isopropanol, 0.34 mL, 1.71 mmol) dropwise. The solution was heated to 40° C. and allowed to proceed overnight. The following morning, the solution was cooled to room temperature, and dry acetonitrile (10 mL) was added to the mixture. The mixture was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((10-aminodecyl)amino)-2-oxoethyl)disulfaneyl)acetate hydrochloride as a white solid (0.05 g, 0.07 mmol, 38.1%). UPLC/ELSD: RT=2.62 min. MS (ES): m/z (MH⁺) 705.9 for C₄₁H₇₃ClN₂O₃S₂. ¹H NMR (300 MHz, MeOD) δ: ppm 5.32 (m, 1H), 4.49 (br. m, 1H), 3.82 (m, 2H), 3.51 (s, 2H), 3.37 (s, 2H), 3.21 (m, 3H), 3.11 (t, 2H), 2.84 (t, 2H), 2.25 (m, 2H), 2.14 (br. m, 1H), 1.94 (br. m, 8H), 1.53 (m, 15H), 1.25 (br. m, 18H), 1.06 (d, 21H, J=6 Hz), 0.96 (m, 8H), 0.86 (d, 6H, J=6 Hz), 0.80 (d, 9H, J=6 Hz), 0.63 (s, 5H).

### Example 2

### Production of Nanoparticle Compositions

Lipid nanoparticles were prepared using ethanol drop nanoprecipitation followed by solvent exchange into suitable aqueous buffer using dialysis. An exemplary lipid nanoparticle composition can be prepared by a process where lipids are dissolved in ethanol at a concentration of 12.5 mM and molar ratios of 33:15:11:39.5:1.5 (e.g., ionizable lipid:SA46:phospholipid:cholesterol:PL1). Lipid to mRNA is maintained at a N/P ratio of 4.9. Then mRNA is diluted with 25 mM sodium acetate (pH 5.0) and combined with the lipid mixture at a volume ratio of 3:1 (aqueous: ethanol). Resulting formulations are dialyzed against 20 mM tris/8% sucrose/70 mM sodium chloride (pH 7.4) at a volume of 300 times that of the primary product using Slide-A-Lyzer dialysis cassettes (Thermo Scientific, Rockford, IL, USA) with a molecular cutoff of 10 KDa for at least 18 h. The first dialysis is carried out at room temperature in a digital orbital shaker (VWR, Radnor, PA, USA) at 85 rpm for 3 h and then dialyzed overnight at 4° C. Formulations are concentrated using Amicon ultra-centrifugal filters (EMD Millipore, Billerica, MA, USA), passed through a 0.22-μm filter and stored at 4° C. until use. Lipid nanoparticle solutions are typically adjusted to specific mRNA concentrations between 0.1 and 1 mg/mL.

### Example 3

### Protein Expression in Human Cervical Cancer Epithelial Cell (HeLa)

LNPs were prepared according to Example 2. To evaluate LNP cellular uptake and protein expression in vitro, HeLa cells from ATCC.org (ATCC CCL-2) are used. The cells are cultured in complete Minimum Essential Medium (MEM) and are plated in 96 well Cell Carrier Ultra plate with PDL coated surface (PerkinElmer) prior to running an experiment.

### Expression Assay in Hela Cells

Buffer control (PBS) and LNPs encapsulating cystic fibrosis transmembrane conductance regulator (CFTR) mRNA were dosed with MEM media in the absence of serum (N=4 replicate wells). LNP transfected cells were incubated for 5 h, followed by media removal and supplementation with complete MEM media. Cells were further incubated in complete MEM media overnight (24 h).

Following 24 hr incubation, the cells were fixed with PFA and processed for immunofluorescence (IF) using an anti-CFTR rabbit monoclonal antibody. Briefly, the cells were permeabilized with 0.5% TX-100 for 10 min, blocked with 3% bovine serum albumin (BSA)+PBST for 1 hr at room temperature, and incubated with primary anti-CFTR monoclonal antibody overnight at 4° C. Following primary antibody incubation, the cells were incubated with Alexa 488 conjugated secondary antibody for 30 min, and stained with DAPI and HCS CellMask Blue stain. Between different incubation steps the cells were either washed with PBS or PBST. Cells were imaged using Opera Phoenix spinning disk confocal microscope (PerkinElmer), and CFTR protein expression was detected using the 488 nm channel. The image analysis was performed in Harmony 4.9, with main analysis output being CFTR intensity per cell. The results shown in Table 3a show fold-change in CFTR signal intensity per cell compared to buffer (PBS) control (nanoparticle composition: IL1:Lipid Amine:DSPC:Chol:PL1). The results shown in Table 3b show fold-change in CFTR signal intensity per cell compared to buffer (PBS) control (nanoparticle composition: IL1:Lipid Amine:DPPC:Chol:PL1).

TABLE 3a

| Lipid | Average fold-change in CFTR signal |
|---|---|
| Amine | intensity per cell ± standard deviation |
| SA54 | 1.5 ± 0.1 |
| SA53 | 4.4 ± 0.8 |
| SA49 | 1.1 ± 0.0 |
| SA47 | 4.6 ± 0.4 |
| SA48 | 4.4 ± 0.3 |
| SA46 | 4.7 ± 0.2 |
| SA55 | 4.9 ± 0.2 |
| SA52 | 2.3 ± 0.1 |

TABLE 3b

| Lipid | Average fold-change in CFTR signal |
|---|---|
| Amine | intensity per cell ± standard deviation |
| SA54 | 1.3 ± 0.1 |
| SA53 | 13.0 ± 1.2 |
| SA49 | 1 ± 0.0 |
| SA47 | 10.9 ± 0.4 |
| SA48 | 5.3 ± 1.0 |
| SA46 | 10.2 ± 0.2 |
| SA55 | 12.1 ± 0.7 |
| SA52 | 9.3 ± 1.2 |

### Example 4

### Production of Nanoparticle Compositions

Lipids were dissolved in ethanol at a concentration of 24 mg/mL and molar ratios of 49.0:11.2:39.3:0.5 (IL1:DSPC:cholesterol:PEG-DMG-2K) and mixed with the acidification buffer (45 mM acetate buffer at pH 4). The lipid solution and acidification buffer were mixed using a multi-inlet vortex mixer at a 3:7 volumetric ratio of lipid:buffer for mixer 1 and mixer 2 and a 1:3 volumetric ratio of lipid:buffer (25% ethanol) for mixer 3. After a 5 second residence time, the resulting nanoparticles were mixed with 55 mM sodium acetate at pH 5.6 at a volumetric ratio of 5:7 of nanoparticle: buffer. See Table 4a for mixing parameters. The resulting dilute nanoparticles were then buffer exchanged and concentrated using tangential flow filtration (TFF) into a final buffer containing 5 mM sodium acetate pH 5.0. See Table 4b for TFF parameters. Then a 70% sucrose solution in 5 mM acetate buffer at pH 5 is subsequently added.

TABLE 4a
Mixing Parameters

| | Mixer | | |
|---|---|---|---|
| Stream | 1 | 2 | 3 |
| 1 mL/min | 21 | 75 | 281 |
| 2 mL/min | 49 | 175 | 844 |
| 3 mL/min | 98 | 350 | 1575 |
| Nano-precipitation | 30% | 30% | 25% |
| (1 + 2) | | | |
| Mixed product | 12.5% | 12.5% | 10.4% |
| (1 + 2 + 3) | | | |

TABLE 4b
TFF Parameters

| TFF Parameter | Mixer Value | |
|---|---|---|
| Feed flux | 240 | L/m²/hr |
| Loading | 100-300 | g/m³ |
| Initial concentration: Factor | 10x | |
| Diavolumes (DVs) | 8 | DVs |
| Final concentration: Factor | 4x | |
| TMP | 4-6 | psi |

The resulting nanoparticles at a lipid concentration of 7.33 mg/mL in 5 mM acetate (pH 5) and 75 g/L sucrose were mixed with mRNA (luciferase or CFTR) at a concentration of 0.625 mg/mL in 42.5 mM sodium acetate pH 5.0, with N: P of 4.93. The nanoparticle solution and nanoparticles were mixed using a multi-inlet vortex mixer at a 3:2 volumetric ratio of nanoparticle:mRNA. Once loaded with mRNA, these intermediate nanoparticles underwent a 300 second residence time prior addition of neutralization buffer containing 120 mM TRIS pH 8.12 at a volumetric ratio of 5:1 of nanoparticle: buffer.

For HeLa studies evaluating CFTR protein expression, the intermediate nanoparticle formulation (1 mL, 0.415 mg mRNA) is mixed with a buffer containing 20 mM TRIS (pH 7.5), 0.9 mg/mL PEG-DMG-2K and lipid amine SA52 (647.1 nmol) at a volumetric ratio of 6:1 of nanoparticle: buffer. The resulting nanoparticle suspension underwent concentration using a centrifugal filtration device (100 kDa molecular weight cut-off) and was diluted in running buffer (20 mM TRIS, 14.3 mM sodium acetate, and 32 g/L sucrose, pH 7.5) with a 300 mM NaCl solution to a final buffer matrix containing 70 mM NaCl.

Both resulting nanoparticle suspensions were filtered through a 0.8/0.2 μm capsule filter and filled into glass vials at an mRNA strength of about 0.1-1 mg/mL Biophysical data (Diameter and PDI from DLS measurements and % Encapsulation using Ribogreen assay) for CFTR mRNA nanoparticles with lipid amines is shown in Table 4c.

TABLE 4c
CFTR mRNA nanoparticle biophysical data

| | | | % Encapsulation |
|---|---|---|---|
| SA# | Diameter (nm) | PDI | (Ribogreen) |
| SA52 | 72.4 | 0.11 | 96.6 |

### Example 5

### Protein Expression in Human Cervical Cancer Epithelial Cell (HeLa)

Lipid nanoparticle compositions were prepared in a manner analogous to that in example 4. To evaluate LNP cellular uptake and protein expression In Vitro, HeLa cells from ATCC.org (ATCC CCL-2) was used. The cells were cultured in complete Minimum Essential Medium (MEM) and were plated in 96 well Cell Carrier Ultra plate with PDL coated surface (PerkinElmer) prior to running an experiment.

### CFTR Protein Expression Assay in Hela Cells

Buffer control (PBS) and LNPs encapsulating cystic fibrosis transmembrane conductance regulator (CFTR) mRNA were dosed with MEM media in the absence of serum (N=4 replicate wells). LNP transfected cells were incubated for 5 hr, followed by media removal and supplementation with complete MEM media. Cells were further incubated in complete MEM media overnight (24 hr).

Following 24 hr incubation, the cells were fixed with PFA and processed for immunofluorescence (IF) using an anti-CFTR rabbit monoclonal antibody. Briefly, the cells were permeabilized with 0.5% TX-100 for 10 min, blocked with 3% bovine serum albumin (BSA)+PBST for 1 hr at room temperature, and incubated with primary anti-CFTR monoclonal antibody overnight at 4° C. Following primary antibody incubation, the cells were incubated with Alexa 488 conjugated secondary antibody for 30 min and stained with DAPI and HCS CellMask Blue stain. Between different incubation steps, the cells were washed with either PBS or PBST. Cells were imaged using Opera Phoenix spinning disk confocal microscope (PerkinElmer), and CFTR protein expression was detected using the 488 nm channel. The image analysis was performed in Harmony 4.9, with main analysis output being CFTR intensity per cell. The results are shown in Table 5a show fold-change in CFTR signal intensity per cell compared to buffer (PBS) control.

TABLE 5a
CFTR Protein Expression Results

| | Average fold-change in CFTR signal intensity per |
|---|---|
| Lipid amine | cell compared to buffer control ± standard deviation |
| SA52 | 3.4 ± 0.5 |

A number of embodiments of the invention have been described. Nevertheless, it will be understood that various modifications may be made without departing from the spirit and scope of the invention. Accordingly, other embodiments are within the scope of the following claims. Each reference, including all patent, patent applications, and publications, cited in the present application is incorporated herein by reference in its entirety.

1. A lipid amine compound of Formula A1: 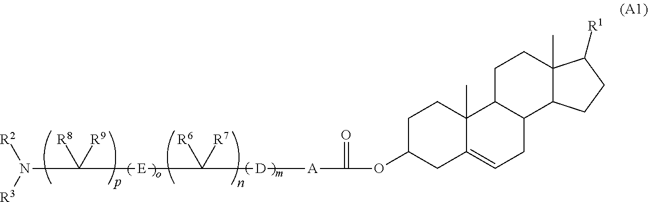
or a salt thereof, wherein:
A is NR^{a} or CR⁴R⁵;
D is O or S—S;
E is C(O), C(O)NH, or O;
R¹ is C₁₋₁₄ alkyl, C₁₋₁₄ alkenyl, or C₁₋₁₄ hydroxyalkyl;
R² and R³ are each independently selected from H, methyl, and ethyl, wherein the methyl or ethyl is optionally substituted by OH;
or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo;
or R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, OH, and halo;
R^{a} is H or methyl;
R⁴, R⁵, R⁶, R⁷, R⁸, R⁹, and R¹⁰ are each independently selected from H and C₁₋₄ alkyl;
or R⁴ and R⁵ together with the carbon atom to which they are attached form a C₃₋₅ cycloalkyl group;
or R⁶ and R⁷ together with the carbon atom to which they are attached form a C₃₋₅ cycloalkyl group;
or R⁸ and R⁹ together with the carbon atom to which they are attached form a C₃₋₅ cycloalkyl group;
m is 0 or 1;
n is 0, 1, 2, 3, 4, or 5;
o is 0 or 1; and
p is 0, 1, 2, 3, 4, 5, 6, 7, 8, 9, 10, 11, or 12;
wherein at least one of m, n, o, and p is other than 0;
wherein p is 1 when R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, OH, and halo; and
wherein when m is 1, then A is CR⁴R⁵ and n is 1,
with the proviso that the compound is other than:

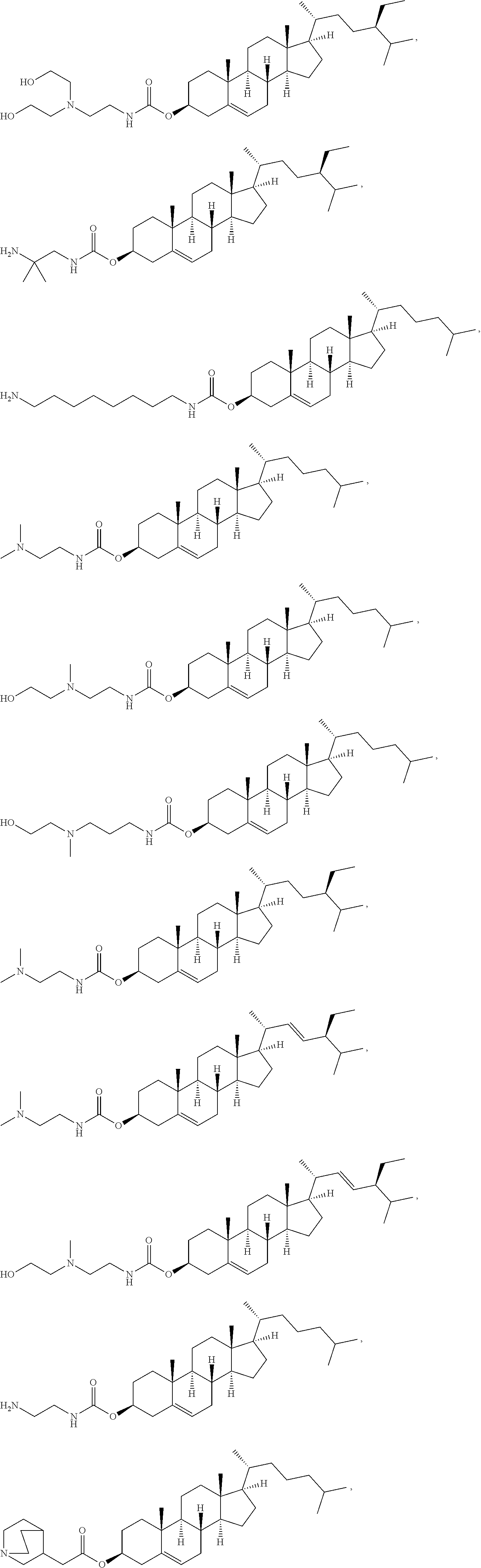

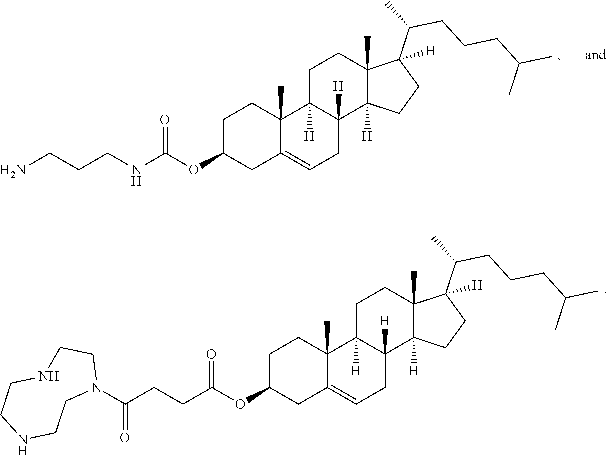

2. The compound of claim 1, or a salt thereof, wherein R¹ is C₁₋₁₄ alkyl.

3. The compound of claim 1, or a salt thereof, wherein R¹ is

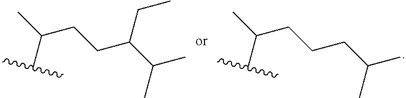

4.-39. (canceled)

40. The compound of claim 1 having Formula A2: 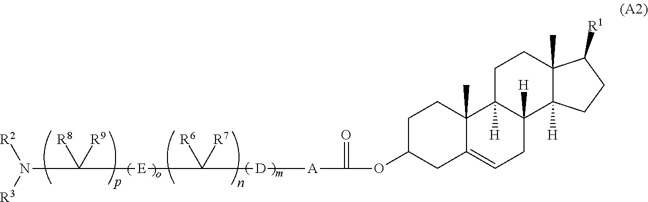
or a salt thereof.

41. The compound of claim 1, or salt thereof, selected from:

| No. | Structure |
|---|---|
| SA44 | 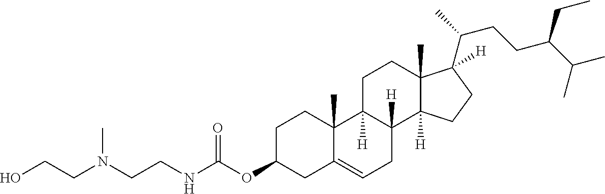 |
| SA45 | 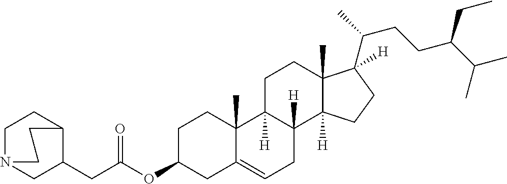 |
| SA46 | 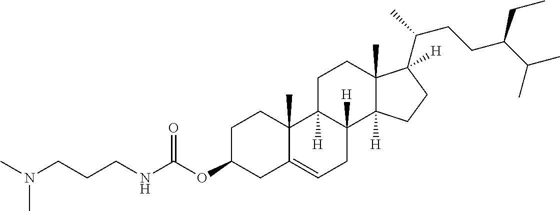 |
| SA47 | 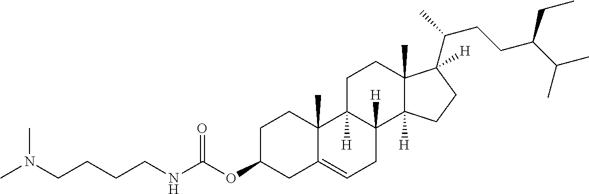 |
| SA49 | 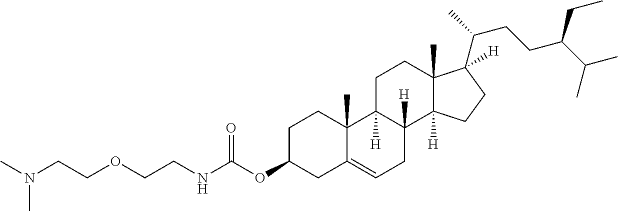 |
| SA52 | 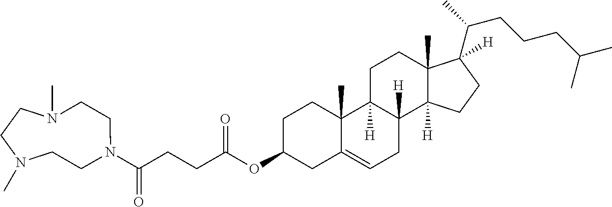 |
| SA53 | 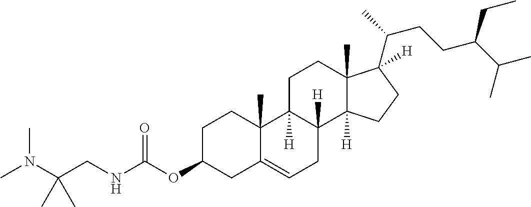 |
| SA54 | 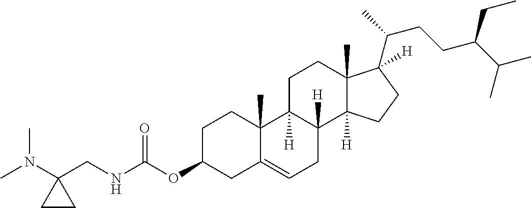 |
| SA86 | 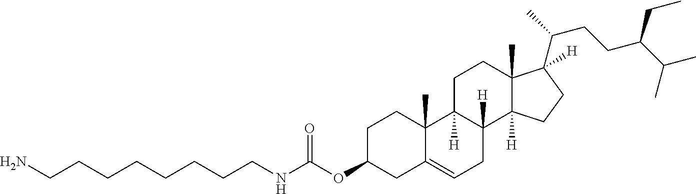 |
| SA91 | 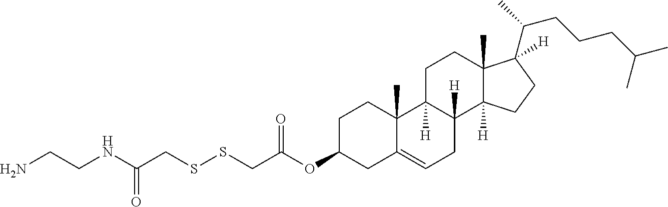 |
| SA92 | 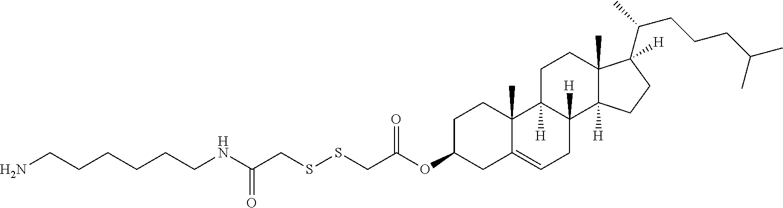 |
| SA93 | 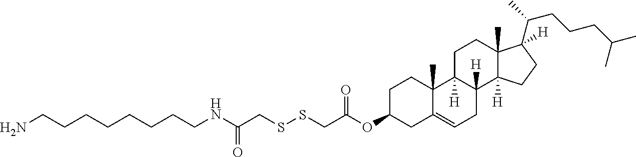 and |
| SA94 | 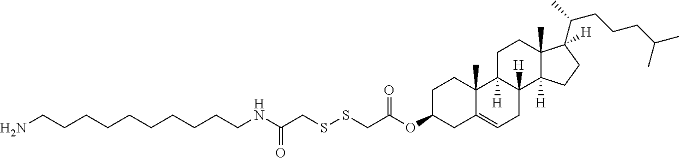 |

42. A lipid nanoparticle composition comprising a lipid amine compound according to claim 1, or a salt thereof.

43. The lipid nanoparticle composition of claim 42, wherein the lipid nanoparticle composition further comprises:
(i) an ionizable lipid,
(ii) a phospholipid,
(iii) a structural lipid,
(iv) optionally a PEG-lipid, and
(v) optionally a payload for delivery into a cell.

44. The lipid nanoparticle composition of claim 43, wherein a weight ratio of the lipid amine to payload is about 1:1 to about 4:1.

45. (canceled)

46. The lipid nanoparticle composition of claim 43, wherein the ionizable lipid is a compound of Formula (I): 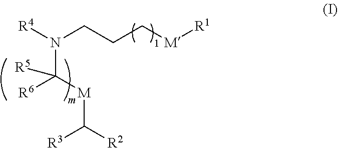
or an N-oxide or a salt thereof, wherein:
R¹ is 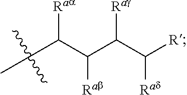
wherein [symbol] denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and 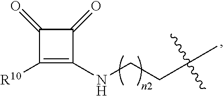
wherein n is selected from 1, 2, 3, 4, and 5;
wherein [symbol] denotes a point of attachment,
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

47. The lipid nanoparticle composition of claim 43, wherein the ionizable lipid is 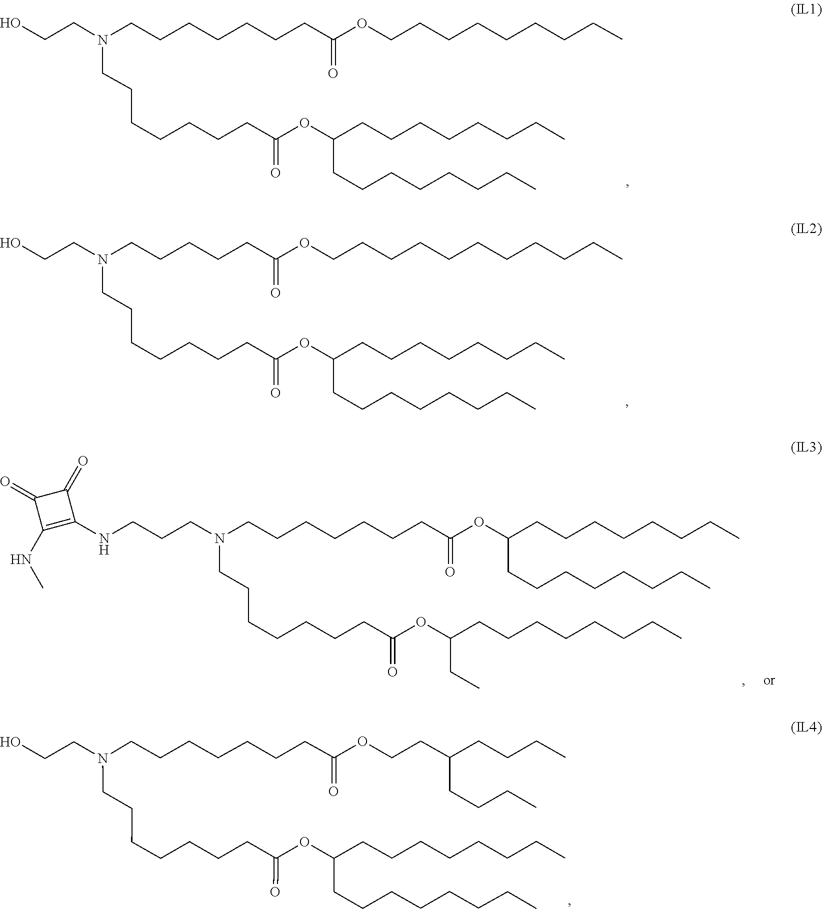 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]
or an N-oxide or a salt thereof.

48. (canceled)

50. The lipid nanoparticle composition of claim 43, wherein the phospholipid is a compound of Formula (IV): 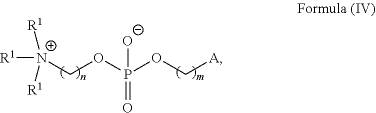
or a salt thereof, wherein:
each R¹ is independently H or optionally substituted alkyl; or optionally two R¹ are joined together with the intervening atoms to form optionally substituted monocyclic cycloalkyl or optionally substituted monocyclic heterocyclyl; or optionally three R¹ are joined together with the intervening atoms to form optionally substituted bicyclic cycloalkyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula: 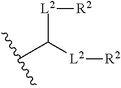
or 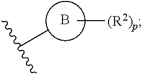
each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2;
provided that the compound is not of the formula: 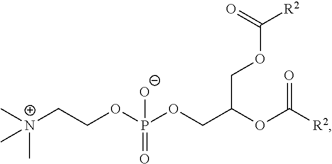
wherein each instance of R² is independently unsubstituted alkyl, unsubstituted alkenyl, or unsubstituted alkynyl.

51. The lipid nanoparticle composition of claim 43, wherein the phospholipid is:
1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-diphytanoyl-sn-glycero-3-phosphoethanolamine (ME 16.0 PE),
1,2-diphytanoyl-sn-glycero-3-phosphocholine (4ME 16:0 PC),
1,2-diphytanoyl-sn-glycero-3-phospho-(1′-rac-glycerol) (sodium salt) (4ME 16:0 PG),
1,2-diphytanoyl-sn-glycero-3-phospho-L-serine (sodium salt) (4ME 16:0 PS),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), or sphingomyelin.

52.-53. (canceled)

54. The lipid nanoparticle composition of claim 43, wherein the structural lipid is cholesterol, fecosterol, sitosterol, ergosterol, campesterol, stigmasterol, brassicasterol, tomatidine, tomatine, ursolic acid, alpha-tocopherol, hopanoids, phytosterols, steroids, or mixtures thereof.

55. The lipid nanoparticle composition of claim 43, wherein the structural lipid is alpha-tocopherol, cholesterol, β-sitosterol, or cholesteryl hemisuccinate.

56. (canceled)

57. The lipid nanoparticle composition of claim 43, wherein the PEG-lipid is a compound of Formula (V): 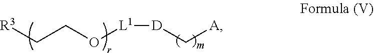
or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted cycloalkylene, optionally substituted heterocyclylene, optionally substituted arylene, optionally substituted heteroarylene, —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
D is a moiety obtained by click chemistry or a moiety cleavable under physiological conditions;
m is 0, 1, 2, 3, 4, 5, 6, 7, 8, 9, or 10;
A is of the formula: 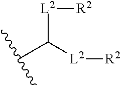
or 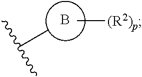
each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(—NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2.

58. The lipid nanoparticle composition of claim 40, wherein the PEG-lipid is PEG-c-DOMG, PEG-DMG, PEG-DLPE, PEG DMPE, PEG-DPPC, PEG-DSPE or PL1:

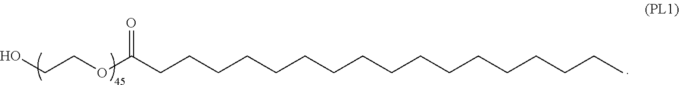

59. (canceled)

60. A pharmaceutical composition comprising the lipid nanoparticle composition of claim 43, or a salt thereof, and a pharmaceutically acceptable carrier.

61. (canceled)

62. A method of delivering a payload into a cell comprising contacting the cell with a lipid nanoparticle composition of claim 43.

63. (canceled)

64. A method of delivering a therapeutic or prophylactic payload to a patient comprising administering a lipid nanoparticle composition of claim 43 to the patient.

65.-68. (canceled)

69. A process of preparing a lipid nanoparticle composition, the process comprising combining the lipid amine compound of claim 1, or a salt thereof, with one or more additional lipids selected from:
(i) an ionizable lipid,
(ii) a phospholipid,
(iii) a structural lipid, and
(iv) a PEG-lipid.

70.-72. (canceled)

73. A lipid nanoparticle composition prepared by the process of claim 69.
